(19) United States
(12) Patent Application Publication
(10) Pub. No.: US 2026/0263661 A1
(43) Pub. Date: Sep. 10, 2026

(54) MULTIPLE FUNCTIONALIZATION OF MULTI-ARM POLYMERS FOR ENHANCED PROPERTIES

(71) Applicant: Boston Scientific Scimed, Inc., Maple Grove, MN (US)

(72) Inventors: Cristian Parisi, Amherst, MA (US); Rick William Dorn, Plymouth, MN (US); Joseph Thomas Delaney, JR., Minneapolis, MN (US)

(73) Assignee: Boston Scientific Scimed, Inc., Maple Grove, MN (US)

(21) Appl. No.: 19/558,966

(22) Filed: Mar. 6, 2026

Related U.S. Application Data
(60) Provisional application No. 63/768,590, filed on Mar. 7, 2025.

(51) Int. Cl. A61L 27/52; A61L 27/18

(52) U.S. Cl. CPC A61L 27/52; A61L 27/18

(57) ABSTRACT

In some aspects, the present disclosure provides multi-arm polymers that comprise a functional core region and three or more polymer arms extending from the functional core region, each polymer arm of the three or more polymer arms comprising a hydrophilic polymer segment, a reactive moiety and a functional moiety, wherein the reactive moiety and the functional moiety are positioned at an end of each polymer arm and are covalently attached to the hydrophilic polymer segment. In some embodiments, the functional core region is an iodinated core region and the functional moiety is an iodinated moiety.

### CROSS-REFERENCE TO RELATED APPLICATIONS

The application claims the benefit of U.S. Provisional Patent Application Ser. No. 63/768,590 filed on Mar. 7, 2025, the disclosure of which is incorporated herein by reference.

### FIELD

The present disclosure relates to functionalized multi-arm polymers, to crosslinked hydrogels formed from such functionalized multi-arm polymers, to crosslinkable systems for forming such crosslinked hydrogels, and to methods of treatment using such crosslinked hydrogels.

### BACKGROUND

SpaceOAR®, a rapid crosslinking hydrogel that polymerizes in vivo within seconds, is based on a multi-arm polyethylene glycol (PEG) polymer with a polyol core functionalized with succinimidyl glutarate as reactive end groups which further react with trilysine to form crosslinks. This product has become a very successful, clinically-used biomaterial in prostate cancer therapy. A further improvement based on this structure is that a portion of the succinimidyl glutarate end groups have been replaced with 2,3,5-triiiodobenzamide groups, providing radiopacity. This hydrogel, known by the trade name of SpaceOAR Vue®, is the radiopaque version of SpaceOAR® for prostate medical applications. Above a specific pH, the succinimidyl glutarate groups of SpaceOAR® and SpaceOAR Vue® will rapidly react with the trilysine crosslinker in vivo to form a hydrogel. The hydrogel breaks down in-vivo over the course of about 6-9 months. The breakdown occurs primarily through the hydrolysis of the ester linkages in the glutarate groups.

The present disclosure provides implantable hydrogel alternatives to SpaceOAR Vue®, thereby expanding the range of medical applications for the hydrogels.

### SUMMARY

In some aspects, the present disclosure provides multi-arm polymers that comprise a functional core region and three or more polymer arms extending from the functional core region, each polymer arm of the three or more polymer arms comprising a hydrophilic polymer segment, a reactive moiety and a functional moiety, wherein the reactive moiety and the functional moiety are positioned at an end of each polymer arm and are covalently attached to the hydrophilic polymer segment.

In some embodiments, the functional core region is an iodinated core region and the functional moiety is an iodinated moiety. In some of these embodiments, the iodinated core region comprises a residue of an iodinated polyhydroxy compound.

In some of these embodiments, which can be used in conjunction with the above aspects and embodiments, the reactive moiety is covalently linked to the hydrophilic polymer segment through a linkage that comprises an ester group.

In some embodiments, which can be used in conjunction with the above aspects and embodiments, the reactive moiety is covalently linked to the hydrophilic polymer segment through a residue of an iodinated compound.

In some embodiments, which can be used in conjunction with the above aspects and embodiments, the reactive moiety is covalently linked to the hydrophilic polymer segment through a residue of an iodinated cyclic anhydride.

In some embodiments, which can be used in conjunction with the above aspects and embodiments, the reactive moiety is covalently linked to the hydrophilic polymer segment through a residue of a cyclic anhydride and a residue of an iodinated hydroxy acid compound.

In some embodiments, which can be used in conjunction with the above aspects and embodiments, the reactive moiety is covalently linked to the hydrophilic polymer segment through a residue of an iodinated amino acid compound and a residue of a cyclic anhydride.

In some embodiments, which can be used in conjunction with the above aspects and embodiments, the reactive moiety is covalently linked to the hydrophilic polymer segment through an amino-alcohol residue.

In some embodiments, which can be used in conjunction with the above aspects and embodiments, the reactive moiety comprises an electrophilic group. In some of these embodiments, the electrophilic group is a cyclic imide group.

In some embodiments, which can be used in conjunction with the above aspects and embodiments, the hydrophilic segment is selected from poly(alkylene oxide) segments, polysaccharide segments, polyoxazoline segments, polydioxanone segments, polypeptide segments, and polyvinyl alcohol segments.

In other aspects, the present disclosure is directed to a system comprising (a) a multi-arm polymer in accordance with the above aspects and embodiments and (b) a multifunctional compound comprising a plurality of reactive groups that are reactive with reactive moieties of the multi-arm polymer. In some of these embodiments, the reactive moieties comprise electrophilic groups and the reactive groups are nucleophilic groups. In some of these embodiments, the electrophilic groups are cyclic imide groups and the nucleophilic groups are amino groups.

In other aspects, the present disclosure is directed to a method of treatment comprising administering to a subject a mixture that comprises (a) a multi-arm polymer in accordance with the above aspects and embodiments and (b) a multifunctional compound comprising a plurality of reactive groups that are reactive with reactive moieties of the multi-arm polymer, under conditions such that the multi-arm polymer and the multifunctional compound crosslink after administration to form a hydrogel.

In some embodiments, the method comprises administering to the subject a first fluid composition that comprises the multi-arm polymer and a second fluid composition that comprises the multifunctional compound.

In some embodiments, the method comprises administering to the subject a first fluid composition that comprises the multi-arm polymer and the multifunctional compound and a second fluid composition that comprises an accelerant that accelerates formation of covalent crosslinks between the multi-arm polymer and the multifunctional compound. In some of these embodiments, the first fluid composition and the second fluid composition are delivered using a double barrel syringe.

In other aspects, the present disclosure is directed to a crosslinked hydrogel composition comprising a crosslinked reaction product of (a) a multi-arm polymer in accordance with the above aspects and embodiments and (b) a multifunctional compound comprising a plurality of reactive groups that are reactive with reactive moieties of the multi-arm polymer, the reactive groups forming covalent bonds with the reactive moieties.

Potential benefits associated with the present disclosure include one or more of the following: overall level of radiocontrast is maintained or enhanced, crosslink density is tunable independently of radiocontrast, and in vivo radiocontrast persistence is obtained.

The above and other aspects, embodiments, features and benefits of the present disclosure will be readily apparent from the following detailed description.

### BRIEF DESCRIPTION OF THE DRAWINGS

FIG. 1A schematically illustrates a multi-arm polymer, in accordance with embodiments of the present disclosure.

FIG. 1B schematically illustrates a multi-arm polymer, in accordance with embodiments of the present disclosure.

FIG. 2 schematically illustrates a delivery device, in accordance with an embodiment of the present disclosure.

FIG. 3 schematically illustrates a delivery device, in accordance with another embodiment of the present disclosure.

### DETAILED DESCRIPTION

In some aspects, the present disclosure provides multi-arm polymers that comprise a functional core region and three or more polymer arms extending from the functional core region, each polymer arm of the three or more polymer arms comprising a hydrophilic polymer segment, a reactive moiety and a functional moiety. The reactive moiety and the functional moiety are positioned at or near an end of each polymer arm and are covalently attached to the hydrophilic polymer segment. The reactive moiety and the functional moiety may be covalently attached to the hydrophilic polymer segment a variety of linkages.

In various embodiments, within each polymer arm, the reactive moiety is covalently attached to the hydrophilic polymer segment through a linkage that comprises a hydrolysable ester group,

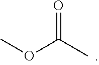

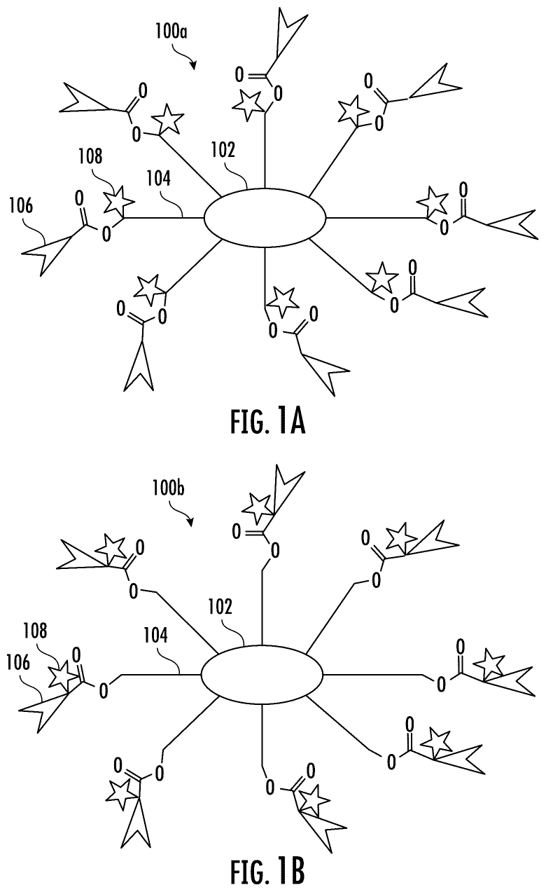

Two particular arrangements of reactive moieties, hydrolysable ester groups, functional moieties, and hydrophilic polymer segments within a multi-arm polymer are illustrated in FIGS. 1A-1B.

With reference to FIG. 1A, a multi-arm polymer 100a is shown which comprises a functional core region 102 from which 8 polymer arms extend. Each polymer arm in FIG. 1A includes a hydrophilic polymer segment 104, a reactive moiety 106, a functional moiety 108, and a hydrolysable ester group. In the multi-arm polymer 100a of FIG. 1A, the hydrolysable ester group is positioned between the reactive moiety 106 and the functional moiety 108, with the reactive moiety 106 positioned on one side of the hydrolysable ester group at an end of the polymer arm, and the functional moiety 108 positioned on an opposite side of the hydrolysable ester group (i.e., on the side of the functional core region 102 and hydrophilic polymer segment 104).

Assuming that the hydrolysable ester groups are the only biodegradable groups in the multi-arm polymer 100a or are the first biodegradable groups in the multi-arm polymer 100a to biodegrade, upon biodegradation, the functional moiety 108 will remain attached to a multi-arm polymer (albeit of reduced size), which contains the functional core region 102 and eight polymer arms, each comprising the hydrophilic polymer segment 104 and the functional moiety 108. The reactive moiety 106 in each arm (or a residue thereof, in the event it has been covalently reacted with a crosslinker to form a crosslinked polymer network as described below) will be detached from the functional moiety 108, the hydrophilic polymer segment 104, and the functional core region 102.

Like FIG. 1A, the multi-arm polymer 100b in FIG. 1B comprises a functional core region 102 from which 8 polymer arms extend, each polymer arm in FIG. 1B including a hydrophilic polymer segment 104, a reactive moiety 106, a functional moiety 108, and a hydrolysable ester group. However, in contrast to FIG. 1A, the hydrolysable ester group is positioned between the functional moiety 108 and the hydrophilic polymer segment 104, with the reactive moiety 106 and the functional moiety 108 positioned on one side of the hydrolysable ester group at or near an end of the polymer arm and the hydrophilic polymer segment 104 and functional core region 102 positioned on an opposite side of the hydrolysable ester group.

Again, assuming that the hydrolysable ester groups are the only biodegradable groups in the multi-arm polymer 100b or are the first biodegradable groups in the multi-arm polymer 100b to biodegrade, upon biodegradation, a reduced-size multi-arm polymer containing the functional core region 102 and eight arms each comprising the hydrophilic polymer segment 104 will be formed. The functional moiety 108 and the reactive moiety 106 (or a residue of the reactive moiety 106, in the event it has been covalently reacted with a crosslinker to form a crosslinked polymer network as described below) in each arm will be detached from hydrophilic polymer segment 104 and functional core region 102.

Although various multi-arm polymers are described below in which the functional core regions are iodinated core regions and in which the functional moieties are iodinated moieties, it will be appreciated that other functional core regions, including core regions containing radiopaque atoms other than iodine, therapeutic-agent-containing core regions, and antimicrobial-agent-containing core regions, and other functional moieties, including moieties containing radiopaque atoms other than iodine, therapeutic-agent-containing moieties and antimicrobial-agent-containing moieties, can be created. The foregoing may be used in any combination. For example, multi-arm polymers can be provided, which contain any combination of the following: (a) a core region selected from an iodinated core region, a core region containing radiopaque atoms other than iodine, a therapeutic-agent-containing core region, and an antimicrobial-agent-containing core region and (b) functional moieties selected from iodinated moieties, moieties containing radiopaque atoms other than iodine, therapeutic-agent-containing moieties, antimicrobial-agent-containing moieties, and any combination of the preceding functional moieties.

Multi-arm polymers in accordance with the present disclosure include polymers having from 3 to 100 arms, for example ranging anywhere from 3 to 4 to 5 to 6 to 7 to 8 to 10 to 12 to 15 to 20 to 25 to 50 to 75 to 100 arms (in other words, ranging between any two of the preceding values).

Hydrophilic polymer segments for the polymer arms of the multi-arm polymers of the present disclosure can be selected from any of a variety of synthetic, natural, or hybrid synthetic-natural polymer segments. Examples of polymer segments include those that are formed from one or more monomers selected from the following: C₁-C₆-alkylene oxides (e.g., ethylene oxide, propylene oxide, tetramethylene oxide, etc.), cyclic ester monomers (e.g. glycolide, lactide, β-propiolactone, β-butyrolactone, γ-butyrolactone, γ-valerolactone, δ-valerolactone, ε-caprolactone, etc.), oxazoline monomers (e.g., oxazoline and 2-alkyl-2-oxazolines, for instance, 2-(C₁-C₆ alkyl)-2-oxazolines, including various isomers, such as 2-methyl-2-oxazoline, 2-ethyl-2-oxazoline, 2-n-propyl-2-oxazoline, 2-isopropyl-2-oxazoline, 2-n-butyl-2-oxazoline, 2-isobutyl-2-oxazoline, 2-hexyl-2-oxazoline, etc.), 2-phenyl-2-oxazoline, polar aprotic vinyl monomers (e.g. N-vinyl pyrrolidone, acrylamide, N-methyl acrylamide, dimethyl acrylamide, N-vinylimidazole, 4-vinylimidazole, sodium 4-vinylbenzenesulfonate, etc.), dioxanone, N-isopropylacrylamide, amino acids and sugars.

Hydrophilic polymer segments for use in the multi-arm polymers of the present disclosure may be selected, for example, from the following polymer segments: polyether segments including poly(C₁-C₆-alkylene oxide) segments such as poly(ethylene oxide) (PEO) (also referred to as polyethylene glycol or PEG) segments, poly(propylene oxide) segments, poly(ethylene oxide-co-propylene oxide) segments, polyester segments including polyglycolide segments, polylactide segments, poly(lactide-co-glycolide) segments, poly(β-propiolactone) segments, poly(β-butyrolactone) segments, poly(γ-butyrolactone) segments, poly(γ-valerolactone) segments, poly(δ-valerolactone) segments, and poly(ε-caprolactone) segments, polyoxazoline segments including poly(2-C₁-C₆-alkyl-2-oxazoline segments) such as poly(2-methyl-2-oxazoline) segments, poly(2-ethyl-2-oxazoline) segments, poly(2-propyl-2-oxazoline) segments, poly(2-isopropyl-2-oxazoline) segments, and poly(2-n-butyl-2-oxazoline) segments, poly(2-phenyl-2-oxazoline) segments, polymer segments formed from one or more polar aprotic vinyl monomers, including poly(N-vinyl pyrrolidone) segments, poly(acrylamide) segments, poly(N-methyl acrylamide) segments, poly(dimethyl acrylamide) segments, poly(N-vinylimidazole) segments, poly(4-vinylimidazole) segments, and poly(sodium 4-vinylbenzenesulfonate) segments, polydioxanone segments, poly(N-isopropylacrylamide) segments, polypeptide segments, and polysaccharide segments.

Hydrophilic polymer segments for use in the multi-arm polymers of the present disclosure typically contain between 5 and 1000 monomer units or more, for example ranging from 5 to 10 to 25 to 50 to 100 to 250 to 500 to 1000 monomer units.

Reactive moieties for use in the multi-arm polymers of the present disclosure include those that comprise electrophilic groups. Electrophilic groups may be selected, for example, from cyclic imide ester groups, such as succinimide ester groups,

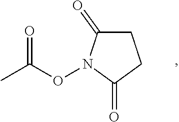

maleimide ester groups, glutarimide ester groups, diglycolimide ester groups, phthalimide ester groups, and bicyclo[2.2.1]hept-5-ene-2,3-dicarboxylic acid imide ester groups,

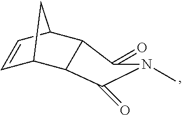

imidazole ester groups, imidazole carboxylate groups and benzotriazole ester groups, among other possibilities.

Iodinated moieties for use in the multi-arm polymers of the present disclosure include those that contain one or more iodinated aromatic groups. Examples of iodinated aromatic groups include iodine-substituted monocyclic aromatic groups and iodine-substituted multicyclic aromatic groups, such as iodine-substituted benzene groups, iodine-substituted naphthylene groups, iodine-substituted anthracene groups, iodine-substituted phenanthrene groups and iodine-substituted tetracene groups, among others. The iodinated aromatic groups may be substituted with one, two, three, four, five, six or more iodine atoms. In some embodiments, the iodinated aromatic groups are further substituted with one or more hydrophilic groups, which may be linked to the aromatic groups directly or may be linked to the aromatic groups through a suitable linking moiety, which may comprise, for example, an amide group, an ether group, and ester group, a carbamate group or a combination thereof, among others. Hydrophilic groups include, for example, acetamido groups that are directedly linked to the aromatic groups, hydroxyl-containing groups including hydroxyl groups that are directly linked to the aromatic groups and hydroxyalkyl groups (e.g., C₁-C₄-hydroxyalkyl groups containing one, two, three or four carbon atoms and containing one, two, three or four or more hydroxyl groups) that are directly linked to the aromatic groups or linked to the aromatic groups through a suitable linking moiety such as those described above. Further examples of iodinated moieties can be found in various embodiments below.

Iodinated core regions for use in the present disclosure include core regions that comprise a residue of an iodinated polyhydroxy compound comprising three or more hydroxyl groups, also referred to herein as an “iodinated polyol”, which is used to form the polymer arms. In certain beneficial embodiments, the core regions comprise residues of iodinated polyols that contains from 3 to 100 hydroxyl groups, for example ranging anywhere from 3 to 4 to 5 to 6 to 7 to 8 to 9 to 10 to 11 to 12 to 15 to 20 to 25 to 50 to 75 to 100 hydroxy groups.

Illustrative iodinated polyols include iodinated aromatic polyols, examples of which are compounds that comprise 3 or more hydroxyl groups, and one or more iodinated aromatic groups. Examples of iodinated aromatic groups include iodine-substituted monocyclic aromatic groups and iodine-substituted multicyclic aromatic groups, such as iodine-substituted benzene groups, iodine-substituted naphthalene groups, iodine-substituted anthracene groups, iodine-substituted phenanthrene groups and iodine-substituted tetracene groups, among others. The aromatic groups may be substituted with one, two, three, four, five, six or more iodine atoms. In various embodiments, the aromatic groups are further substituted with two or more hydroxyl-containing groups, including hydroxyl groups that are directly linked to the aromatic groups and hydroxyalkyl groups (e.g., C₁-C₄-hydroxyalkyl groups containing one, two, three or four carbon atoms and containing one, two, three or four or more hydroxyl groups), which may be linked to the aromatic group directly or through any suitable linking moiety, which may be selected, for example, from amide groups, ester groups, ether groups, carbamate groups, and combinations thereof, among others. Several specific iodinated polyols, along with the CAS numbers of the polyols and the number of hydroxyl groups in the polyols, are listed in the following table:

| | | # of —OH |
|---|---|---|
| Compound | CAS # | Groups |
| 1,3,5-triiodo-2,4,6-tris-hydroxymethylbenzene | 126688-84-2 | 3 |
| Iopromide | 73334-07-3 | 4 |
| 5-Acetamido-N,N′-bis(2,3-dihydroxypropyl)-2,4,6- | 31127-80-7 | 4 |
| triiodoisophthalamide | | |
| Metrizamide | 31112-62-6 | 4 |
| 1,3-Benzenedicarboxamide,5-(acetylamino)-N1,N3- | 76350-28-2 | 4 |
| bis(2,3-dihydroxypropyl)-2,4,6-triiodo-N1-methyl- | | |
| (ACI) | | |
| Iopamidol | 60166-93-0 | 5 |
| Iomeprol | 78649-41-9 | 5 |
| 1,3-Benzenedicarboxamide,N1,N3-bis[2-hydroxy-1- | 60208-45-9 | 5 |
| (hydroxymethyl)ethyl]-5-[(2-hydroxy-1- | | |
| oxopropyl)amino]-2,4,6-triiodo-(ACI) | | |
| Ioxilan | 107793-72-6 | 5 |
| Iopentol | 89797-00-2 | 5 |
| Ioversol | 87771-40-2 | 6 |
| Iohexol | 66108-95-0 | 6 |
| Iobitridol | 136949-58-1 | 6 |
| Iodixanol | 92339-11-2 | 9 |
| 1,3-Benzenedicarboxamide,5,5′-[(1,3-dioxo-1,3- | 79770-24-4 | 12 |
| propanediyl)bis(methylimino)]bis[N,N′-bis[2,3- | | |
| dihydroxy-1-(hydroxymethyl)propyl]-2,4,6-triiodo- | | |
| (9CI, ACI) | | |

Further various iodinated polyols can be found, for example, in U.S. Ser. No. 18/762,216, the disclosure of which is hereby incorporated by reference. These include various iodinated polyols that contain 8 hydroxyl groups and contain four 1,3,5-triiodo-2,4-dihydroxymethyl benzene groups such as the following:

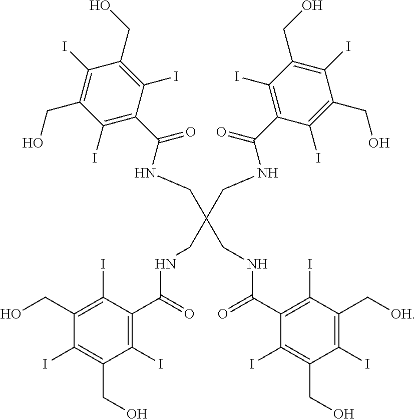 [SMILES: O=C(NCC(CNC(=O)c1c(I)c(CO)c(I)c(CO)c1I)(CNC(=O)c1c(I)c(CO)c(I)c(CO)c1I)CNC(=O)c1c(I)c(CO)c(I)c(CO)c1I)c1c(I)c(CO)c(I)c(CO)c1I]

In some embodiments of the present disclosure, an iodinated polyol such as one of those described above, among others, may be used to form a multi-functional initiator for polymer chain growth.

For example, in some embodiments, an iodinated polyol may be used as an initiator for ring-opening polymerization of ethylene oxide to form polyethylene oxide (PEO) segments (also referred to a polyethylene glycol (PEG) segments) at a position of each of the hydroxyl groups of the polyol. The resulting hydroxyl-terminated PEG segments possess tunable hydrophilicity depending on the desired water-solubility of the resulting multi-arm polymer, for example, with increasing PEG segment length leading to increasing hydrophilicity in some embodiments.

In one particular embodiment, an iodinated polyol, specifically, 1,3,5-triiodo-2,4,6-tris-hydroxymethylbenzene may be used as an initiator, which undergoes ring-opening polymerization with ethylene oxide. The polymerization process leads to poly(ethylene oxide) (PEG) chain growth at each of the three hydroxyl groups of the 1,3,5-triiodo-2,4,6-tris-hydroxymethylbenzene. The resulting multi-arm polymer,

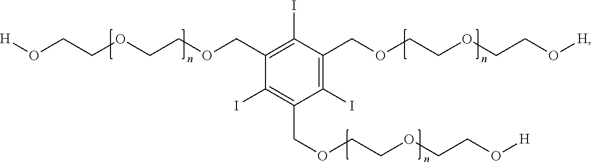

where n is an integer and may range, for example, from 5 to 500, contains three PEG arms that extend from a 1,3,5-triiodo-2,4,6-tris-hydroxymethylbenzene residue core. Each of the PEG arms has a terminal hydroxyl group. Analogous methods can be performed with other iodinated polyols including those described above. For example, a hydroxy-terminated polymer eight-arm polymer,

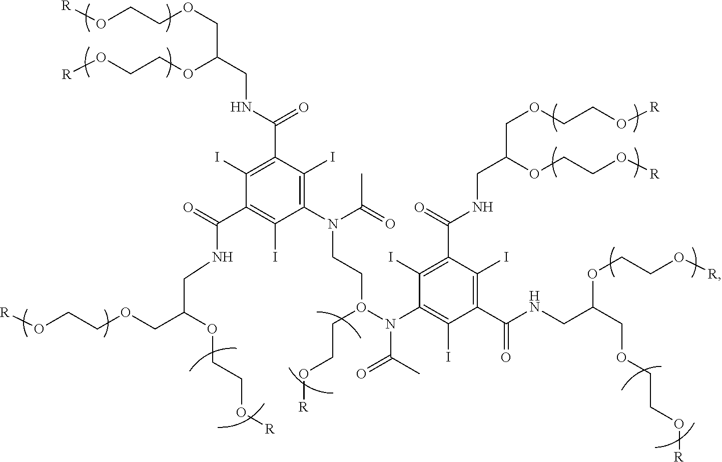

can be formed using iodixanol as the iodinated polyol, where the parentheses designate ethylene oxide segments, which can contain, for example from 5 to 500 ethylene oxide units.

As another example, an iodinated polyol may be reacted with methanesulfonyl chloride to convert the hydroxyl groups into methanesulfonate groups, which can serve as leaving groups for the cationic polymerization of an oxazoline monomer to form polyoxazoline segments at the site of each of the hydroxyl groups of the polyol. The resulting hydroxyl-terminated polyoxazoline segments possess tunable hydrophilicity depending on the desired water-solubility of the resulting multi-arm polymer, for example, with increasing polyoxazoline segment length leading to increased hydrophilicity in some embodiments.

In one particular embodiment, an iodinated polyol, for example, 1,3,5-triiodo-2,4,6-tris-hydroxymethylbenzene is reacted with methanesulfonyl chloride to convert the hydroxyl groups of the 1,3,5-triiodo-2,4,6-tris-hydroxymethylbenzene into methanesulfonate groups, thereby forming a tri-functional initiator, which can be used for the polymerization of 2-ethyl-2-oxazoline. The polymerization process leads to poly(2-ethyl-2-oxazoline) chain growth at sites previously occupied by each of the three hydroxyl groups of the 1,3,5-triiodo-2,4,6-tris-hydroxymethylbenzene, forming a hydroxyl-terminated 3-arm-poly(2-ethyl-2-oxazoline) having a 1,3,5-triiodo-2,4,6-tris-hydroxymethylbenzene residue core and three poly(2-ethyl-2-oxazoline) arms. Each of the polyoxazoline arms has a terminal hydroxyl group. Analogous methods can be performed with other iodinated polyols including those described above.

Further examples of polymer chain grown from iodinated polyols can be found, for example, in U.S. Ser. Nos. 18/762,216 and 18/979,225, the disclosures of which are hereby incorporated by reference.

Using these and other strategies, a variety of multi-arm polymers having a variety of iodinated polyol residue cores and a variety of hydroxy-terminated polymer arms may be provided.

Various multi-arm polymers in accordance with the present disclosure can be formed from iodinated hydroxyl-terminated multi-arm polymers such as those described above through various synthetic routes, including those described below.

As seen above in conjunction with FIGS. 1A-1B, multi-arm polymers in accordance with the present disclosure include those with polymer arms in which a reactive moiety is linked to a hydrophilic polymer segment through a linkage that comprises an ester group.

In some of these embodiments, the iodinated moiety is formed from an iodinated compound, and the reactive moiety is covalently linked to the hydrophilic polymer segment through a residue of the iodinated compound.

In some of these embodiments, the iodinated moiety is formed from an iodinated cyclic anhydride compound, and the reactive moiety is covalently linked to the hydrophilic polymer segment through a residue of the iodinated cyclic anhydride. In these embodiments the multi-arm polymers have polymer arms in which an iodinated cyclic anhydride residue is covalently linked to the hydrophilic polymer segment and in which a reactive moiety is covalently linked to the iodinated cyclic anhydride residue.

Iodinated cyclic anhydrides include cyclic anhydrides in which at least one ring carbon of a cyclic anhydride compound is substituted with iodine alone or an iodinated moiety. Examples include cyclic anhydride compounds in which at least one ring carbon is substituted with an iodinated moiety that comprises an iodinated aromatic group. Examples of iodinated aromatic groups include iodine-substituted monocyclic aromatic groups and iodine-substituted multicyclic aromatic groups, such as iodine-substituted benzene groups, iodine-substituted naphthalene groups, iodine-substituted anthracene groups, iodine-substituted phenanthrene groups and iodine-substituted tetracene groups, among others. The iodinated aromatic groups may be substituted with one, two, three, four, five, six or more iodine atoms. In various embodiments, the aromatic groups may be further substituted with one or more hydrophilic groups, for example, one, two, three, four, five, six or more hydrophilic groups. The hydrophilic groups may be hydroxyl-containing groups, which may be selected, for example, from hydroxyl groups and hydroxyalkyl groups (e.g., hydroxyalkyl groups containing one carbon, two carbons, three carbons, four carbons, etc.). The iodinated aromatic groups may be directly linked to the ring carbon or may be linked to the ring carbon through any suitable linking moiety, which may be selected, for example, from an alkyl group, ether group, ester group, amide group, amine group, or carbonate group, among others.

Specific examples of iodinated aromatic groups include those that comprise one or more monocyclic or multicyclic aromatic structures, substituted with (a) one or more iodine groups (e.g., one two, three, four, five, six or more iodine atoms) and (b) optionally, one or more hydroxyl-containing groups independently selected from one or more hydroxyl groups and/or one or more C₁-C₄-hydroxyalkyl groups (e.g., C₁-C₄-monohydroxyalkyl groups, C₁-C₄-dihydroxyalkyl groups, C₁-C₄-trihydroxyalkyl groups, C₁-C₄-tetrahydroxyalkyl groups, etc.), among others, which C₁-C₄-hydroxyalkyl groups may be linked to the monocyclic or multicyclic aromatic structures directly or through any suitable linking moiety, which may be selected, for example, from alkyl groups, ether groups, ester groups, amide groups, amine groups, or carbonate groups, among others.

In other embodiments, the iodinated cyclic anhydrides are bicyclic structures containing a cyclic anhydride ring in which two carbon atoms are shared with an iodinated aromatic ring, examples of which include phthalic-anhydride-based compounds in which the aromatic ring is substituted with one or more iodine groups.

Some specific examples of iodinated cyclic anhydrides for use in the present disclosure include the following: iodinated glutaric anhydride compounds, including 4-(2,3,5-triiodophenyl)tetrahydropyran-2,6-dione,

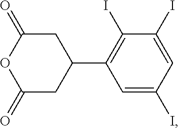 [SMILES: O=C1CC(c2cc(I)cc(I)c2I)CC(=O)O1]

4-(2-iodophenyl)tetrahydropyran-2,6-dione, 4-(3-iodophenyl)tetrahydropyran-2,6-dione, 4-(4-iodophenyl)tetrahydropyran-2,6-dione, 4-((4-iodophenyl)methyl), tetrahydropyran-2,6-dione,

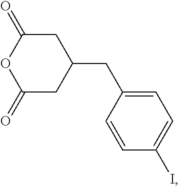 [SMILES: O=C1CC(Cc2ccc(I)cc2)CC(=O)O1]

and 4-1(2-iodophenyl)methyl, tetrahydropyran-2,6-dione; iodinated succinic anhydride compounds, including, 3-(2-iodophenyl)tetrahydrofuran-2,5-dione,

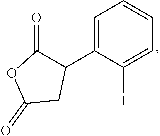 [SMILES: O=C1CC(c2ccccc2I)C(=O)O1]

3-(3-iodophenyl)tetrahydrofuran-2,5-dione, 3-(4-iodophenyl)tetrahydrofuran-2,5-dione, 3-1(2-iodophenyl)methyltetrahydrofuran-2,5-dione,

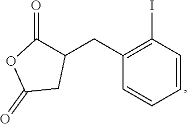 [SMILES: O=C1CC(Cc2ccccc2I)C(=O)O1]

and 3-((4-iodophenyl)methyl)tetrahydrofuran-2,5-dione; and iodophthalic anhydride compounds such as 4,5,7-triiodoisobenzofuran-1,3-dione,

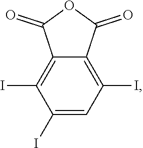 [SMILES: O=C1OC(=O)c2c(I)c(I)cc(I)c21]

4,5,6-triiodoisobenzofuran-1,3-dione, 4,6-diiodoisobenzofuran-1,3-dione, 4,5-diiodoisobenzofuran-1,3-dione, 5,6-diiodoisobenzofuran-1,3-dione, 4,7-diiodoisobenzofuran-1,3-dione, 5-iodoisobenzofuran-1,3-dione, and 4-iodoisobenzofuran-1,3-dione, among others.

Further examples iodinated cyclic anhydrides, and reactive multi-arm polymers formed using the same, can be found, for example, in US 2024/0218122A1 and US 2024/0366503A1, the disclosures of which are hereby incorporated by reference.

The preceding iodinated cyclic anhydrides, among others, may be reacted with a hydroxy-terminated multi-arm polymer such as is described above under basic conditions to form an iodinated carboxylic-acid-terminated precursor polymer comprising a carboxylic acid end group that is linked to a hydrophilic polymer segment through a hydrolysable ester group.

A reactive moiety, for example, an electrophilic moiety, may then be linked to the iodinated carboxylic-acid-terminated precursor polymer. For example, an N-hydroxy cyclic imide compound (e.g., N-hydroxysuccinimide, N-hydroxymaleimide, N-hydroxyglutarimide, N-hydroxyphthalimide, etc.) may be reacted with the iodinated carboxylic-acid-terminated precursor polymer in the presence of a suitable coupling agent (e.g., a carbodiimide coupling agent such as N,N′-dicyclohexylcarbodiimide (DCC), 1-ethyl-3-(3-dimethyl′propyl)carbodiimide (EDC), N-hydroxybenzotriazole (HOBt), BOP reagent, and/or another coupling agent) to form a reactive cyclic imide ester group containing one or more iodine atoms (e.g., an iodinated succinimide ester group, an iodinated maleimide ester group, an iodinated glutarimide ester group, an iodinated phthalimide ester group, etc.) that is linked to a hydrophilic polymer segment through a hydrolysable ester group. In this way, a number of reactive iodinated diester groups can be formed.

For example, in the particular case of N-hydroxysuccinimide as an N-hydroxy cyclic imide compound, exemplary reactive end groups include iodinated succinimidyl malonate groups, iodinated succinimidyl glutarate groups, iodinated succinimidyl succinate groups, iodinated succinimidyl adipate groups, iodinated succinimidyl diglycolate groups, and iodinated succinimidyl phthalate groups, among others. In the particular case of N-hydroxymaleimide as an N-hydroxy cyclic imide compound, exemplary reactive end groups include iodinated maleimidyl malonate groups, iodinated maleimidyl glutarate groups, iodinated maleimidyl succinate groups, iodinated maleimidyl adipate groups, iodinated maleimidyl diglycolate groups, and iodinated maleimidyl phthalate groups, among others. In the particular case of N-hydroxyglutarimide as an N-hydroxy cyclic imide compound, exemplary reactive end groups include iodinated glutarimidyl malonate groups, iodinated glutarimidyl glutarate groups, iodinated glutarimidyl succinate groups, iodinated glutarimidyl adipate groups, iodinated glutarimidyl diglycolate groups, and iodinated glutarimidyl phthalate groups, among others. In the particular case of N-hydroxyphthalimide as an N-hydroxy cyclic imide compound, exemplary reactive end groups include iodinated phthalimidyl malonate groups, iodinated phthalimidyl glutarate groups, iodinated phthalimidyl succinate groups, iodinated phthalimidyl adipate groups, iodinated phthalimidyl diglycolate groups, and iodinated phthalimidyl phthalate groups, among others.

In a particular embodiment, a hydroxy-terminated 3-arm-PEG such as that described above may be used as a tri-functional macro-initiator to react with 4-(2,3,5-triiodophenyl)tetrahydropyran-2,6-dione via ring opening to produce an iodinated carboxyl-terminated 3-arm polymer having a 1,3,5-triiodo-2,4,6-tris-hydroxymethylbenzene residue core and three polymer arms, each polymer arm comprising a hydrophilic PEG segment linked to the core and a carboxy-terminated iodinated glutaric acid residue linked by an ester bond to each hydrophilic PEG segment. The iodinated glutaric acid end-capped 3-arm PEG is illustrated here,

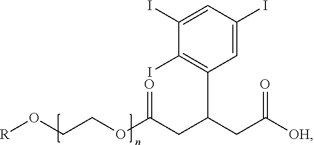

where n is an integer and may have a value ranging from 5 to 500, and R represents the 1,3,5-triiodo-2,4,6-tris-hydroxymethylbenzene residue core and the remaining two polymer arms.

In another embodiment, a hydroxy-terminated 3-arm-PEG such as that described above may be reacted with 3,4,5,6-tetraiodophthalic anhydride in a ring-opening reaction to produce an iodinated carboxyl-terminated 3-arm polymer having a 1,3,5-triiodo-2,4,6-tris-hydroxymethylbenzene residue core and three polymer arms, each polymer arm comprising a hydrophilic PEG segment linked to the core and a carboxy-terminated 3,4,5,6-tetraiodophthalic anhydride residue linked by an ester bond to each hydrophilic PEG segment. The tetraiodophthalic-anhydride-residue-end-capped 3-arm PEG is illustrated here,

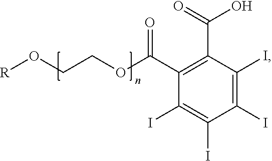

where n is an integer and may have a value ranging from 5 to 500, and R represents the 1,3,5-triiodo-2,4,6-tris-hydroxymethylbenzene residue core and the remaining two polymer arms.

Once an iodinated-anhydride-residue-end-capped multi-arm polymer is formed, a reactive moiety, such as an electrophilic moiety, may then be linked to the carboxyl groups of the iodinated-anhydride-residue-end-capped multi-arm polymer as previously described.

In a particular embodiment, the above-described iodinated glutaric acid end-capped 3-arm PEG is reacted with N-hydroxysuccinimide in the presence of a suitable coupling agent to form an iodinated succinimidyl glutarate end-capped 3-arm PEG polymer,

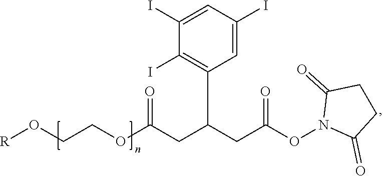

where n is an integer and may have a value ranging from 5 to 500, and R represents the 1,3,5-triiodo-2,4,6-tris-hydroxymethylbenzene residue core and the remaining two polymer arms.

In another particular embodiment, the above-described tetraiodophthalic-anhydride-residue-end-capped 3-arm PEG is reacted with N-hydroxysuccinimide in the presence of a coupling agent to form a succinimidyl-ester-substituted, tetraiodophthalic-anhydride-residue-end-capped 3-arm PEG,

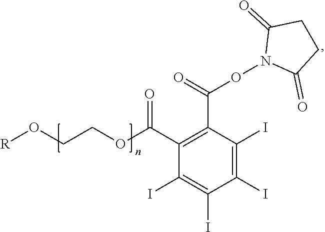

where n is an integer and may have a value ranging from 5 to 500, and R represents the 1,3,5-triiodo-2,4,6-tris-hydroxymethylbenzene residue core and the remaining two polymer arms.

Using these and other strategies, a variety of multi-arm polymers having a variety of iodinated polyol residue cores and a variety of cyclic-imide-ester-terminated polymer arms, in which the cyclic imide ester is attached to a variety of polymer segments through a variety of iodinated cyclic anhydride residues, may be provided.

With reference again to FIG. 1B, it can be seen that the preceding multi-arm polymers fall into the category wherein the reactive moiety 106 and the functional/iodinated moiety 108 are positioned on one side of the hydrolysable ester group at an end of the polymer arm and the hydrophilic polymer segment 104 and the functional/iodinated core region 102 are positioned on an opposite side of the hydrolysable ester group.

As previously noted, in various embodiments, the iodinated moiety of the multi-arm polymer is formed from an iodinated compound, and the reactive moiety is covalently linked to the hydrophilic polymer segment of the multi-arm polymer through an iodinated residue of the iodinated compound.

In some of these embodiments, the iodinated moiety is formed from an iodinated hydroxy acid compound, and the reactive moiety is covalently linked to the hydrophilic polymer segment through a residue of the iodinated hydroxy acid compound and a residue of a cyclic anhydride. In these embodiments the multi-arm polymers have polymer arms in which an iodinated hydroxy acid compound residue is covalently linked to the hydrophilic polymer segment and in which a reactive moiety is covalently linked to the iodinated hydroxy acid compound residue through a cyclic anhydride.

Iodinated hydroxy acid compounds for use herein contain a carboxyl group (also referred to herein as a carboxylic acid group) and one or more hydroxyl groups (also referred to herein as hydroxy groups). Examples of iodinated hydroxy acid compounds include those that comprise at least one iodinated aromatic group (e.g., a monocyclic or multicyclic aromatic structure that is substituted with one, two, three, four, five, six or more iodine atoms), a carboxylic acid group, and one or more hydroxyl-containing groups. Hydroxyl-containing groups include hydroxyl groups that are directly linked to the least one iodinated aromatic group and hydroxyalkyl groups (e.g., C₁-C₄-hydroxyalkyl groups containing one, two, three or four carbon atoms and containing one, two, three or four or more hydroxyl groups) that are directly linked to the least one iodinated aromatic groups or linked to the least one iodinated aromatic groups through a suitable linking moiety, which may comprise, for example, an amide group, an ether group, and ester group, a carbamate group or a combination thereof, among others.

Specific examples of iodinated hydroxy acid compounds include the following, among many others: hydroxymethyl iodobenzoic acid,

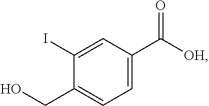 [SMILES: O=C(O)c1ccc(CO)c(I)c1]

hydroxymethyl diiodobenzoic acid,

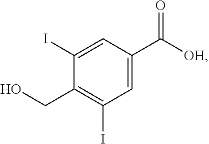 [SMILES: O=C(O)c1cc(I)c(CO)c(I)c1]

4, 4-bis(hydroxy-3, 5-diidophenyl) pentanoic acid (IBHP),

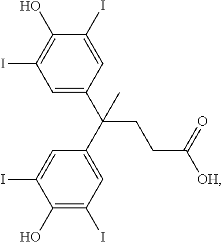 [SMILES: CC(CCC(=O)O)(c1cc(I)c(O)c(I)c1)c1cc(I)c(O)c(I)c1]

N-acetyl-3,5-diiodo-L-tyrosine,

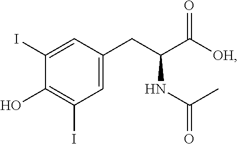 [SMILES: CC(=O)N[C@@H](Cc1cc(I)c(O)c(I)c1)C(=O)O]

N-acetyl-3-diiodo-L-tyrosine,

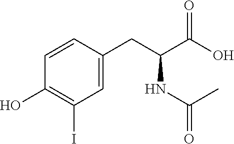 [SMILES: CC(=O)N[C@@H](Cc1ccc(O)c(I)c1)C(=O)O]

and N-acteyl-thyroxine,

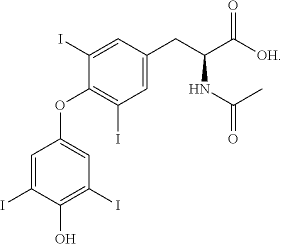 [SMILES: CC(=O)N[C@@H](Cc1cc(I)c(Oc2cc(I)c(O)c(I)c2)c(I)c1)C(=O)O]

Various iodinated hydroxy acid compounds are described in US 2024/0189485, the disclosure of which is hereby incorporated by reference.

The preceding iodinated hydroxy acid compounds, among others, may be coupled with a hydroxy-terminated multi-arm polymer, such as those described above among others, via an ester coupling reaction between the carboxyl groups of the iodinated hydroxy acid compound and the hydroxyl groups of the hydroxy-terminated multi-arm polymer to produce an iodinated hydroxy-terminated multi-arm polymer in which residues of the iodinated hydroxy acid compound are attached to the hydrophilic polymer segments of the polymer arms through an ester linkage. Such reactions may be performed in the presence of a suitable ester coupling agent. Examples of suitable ester coupling agents include carbodiimide coupling agents such as N,N′-dicyclohexylcarbodiimide (DCC), 1-ethyl-3-(3-dimethyl′propyl)carbodiimide (EDC), and 1,3-diisopropylcarbodiimide (DIC), N-hydroxybenzotriazole (HOBt), BOP reagent, and TBTU (2-(1H-Benzotriazole-1-yl)-1,1,3,3-tetramethylaminium tetrafluoroborate), among others.

In a particular embodiment, an iodinated hydroxy-terminated 3-arm-PEG such as that described above may be reacted in an ester coupling reaction with hydroxymethyl iodobenzoic acid to produce an iodinated hydroxyl-terminated 3-arm polymer having a 1,3,5-triiodo-2,4,6-tris-hydroxymethylbenzene residue core and three polymer arms, each polymer arm comprising a hydrophilic PEG segment linked to the core and an iodinated hydroxymethyl iodobenzoic acid residue linked by an ester bond to each hydrophilic PEG segment. The iodinated hydroxyl-terminated 3-arm PEG is illustrated here, where n is an integer and may have a value ranging from 5 to 500, and R represents the 1,3,5-triiodo-2,4,6-tris-hydroxymethylbenzene residue core and the remaining two polymer arms:

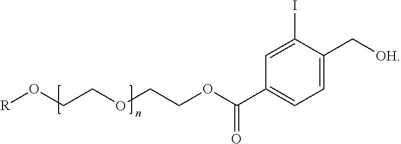

In various embodiments, hydroxyl groups of the iodinated hydroxy-terminated multi-arm polymer are reacted in a ring-opening reaction with an iodinated or non-iodinated cyclic anhydride (e.g., glutaric anhydride, succinic anhydride, malonic anhydride, adipic anhydride, diglycolic anhydride, etc.) to form a reaction product that comprises carboxylic acid end groups that are linked to the residue of the iodinated hydroxy acid compound through an ester linkage. Various anhydrides, along with their CAS numbers, some of which are iodinated, are listed in the following table.

| Species | CAS # |
|---|---|
| Succinic Anhydride | 108-30-5 |
| Glutaric Anhydride | 108-55-4 |
| Adipic Anhydride | 2035-75-8 |
| 2,8-Oxocanedione | 10521-07-0 |
| 3-Oxabicyclo[3.1.0]hexane-2,4-dione | 5617-74-3 |
| 3-Oxabicyclo[3.2.0]heptane-2,4-dione | 4462-96-8 |
| Dihydro-4-methyl-2H-pyran-2,6(3H)-dione | 4166-53-4 |
| Itaconic anhydride | 2170-03-8 |
| 2,2-Dimethylsuccinic anhydride | 17347-61-4 |
| 6,6-Dimethyl-3-oxabicyclo[3.1.0]hexane-2,4-dione | 5466-84-2 |
| Dihydro-4,4-dimethyl-2H-pyran-2,6(3H)-dione | 4160-82-1 |
| Diglycolic anhydride | 4480-83-5 |
| 2H-Pyran-2,4,6(3H,5H)-trione | 10521-08-1 |
| Dihydro-4-(2-methylpropyl)-2H-pyran-2,6(3H)-dione | 185815-59-2 |
| 3-Iodophthalic anhydride* | 28418-88-4 |
| Tetraiodophthalic anhydride* | 632-80-4 |
| 5-Iodo-1,3-isobenzofurandione* | 28418-89-5 |
| 5,6-Diiodo-1,3-isobenzofurandione* | 25834-17-7 |
| Dihydro-3-(2-iodophenyl)-2,5-furandione* | 887131-98-8 |

*Iodinated Compounds.

A reactive moiety, for example, an electrophilic moiety, may be linked to the carboxyl groups of the iodinated carboxyl-terminated multi-arm polymer that is produced by the above-described ring-opening reaction. For example, an N-hydroxy cyclic imide compound (e.g., N-hydroxysuccinimide, N-hydroxymaleimide, N-hydroxyglutarimide, N-hydroxyphthalimide, etc.) may be reacted with the carboxylic acid groups in the presence of a suitable amide coupling agent (e.g., a carbodiimide coupling agent such as N,N′-dicyclohexylcarbodiimide (DCC), 1-ethyl-3-(3-dimethyl′propyl)carbodiimide (EDC), N-hydroxybenzotriazole (HOBt), BOP reagent, and/or another coupling agent) to form a reactive cyclic imide ester end group that is linked to a residue of an iodinated hydroxy acid compound through an ester linkage. In this way, a number of reactive diester end groups can be formed.

For example, in the particular case of N-hydroxysuccinimide as an N-hydroxy cyclic imide compound, exemplary reactive diester end groups include succinimidyl malonate groups, succinimidyl glutarate groups, succinimidyl succinate groups, succinimidyl adipate groups, and succinimidyl diglycolate groups, among others. In the particular case of N-hydroxymaleimide as an N-hydroxy cyclic imide compound, exemplary reactive diester end groups include maleimidyl malonate groups, maleimidyl glutarate groups, maleimidyl succinate groups, maleimidyl adipate groups, and maleimidyl diglycolate groups, among others. In the particular case of N-hydroxyglutarimide as an N-hydroxy cyclic imide compound, exemplary reactive diester end groups include glutarimidyl malonate groups, glutarimidyl glutarate groups, glutarimidyl succinate groups, glutarimidyl adipate groups, glutarimidyl diglycolate groups, among others. In the particular case of N-hydroxyphthalimide as an N-hydroxy cyclic imide compound, exemplary reactive diester end groups include phthalimidyl malonate groups, phthalimidyl glutarate groups, phthalimidyl succinate groups, phthalimidyl adipate groups, and phthalimidyl diglycolate groups, among others.

Using these and other strategies, a variety of multi-arm polymers having a variety of iodinated polyol residue cores and a variety of cyclic-imide-ester-terminated polymer arms, in which the cyclic imide ester is attached to a variety of polymer segments through a variety of cyclic anhydride residues and a variety iodinated hydroxy acid compound residues, may be provided.

With reference again to FIG. 1B, it can be seen that the preceding multi-arm polymers fall into the category wherein the reactive moiety 106 and the functional/iodinated moiety 108 are positioned on one side of a hydrolysable ester group at an end of the polymer arm and the hydrophilic polymer segment 104 and the functional/iodinated core region 102 positioned on an opposite side of the hydrolysable ester group. In addition, the reactive moiety 106 is attached to the functional/iodinated moiety 108 through a linkage that includes an additional hydrolysable ester group.

As previously noted, in various embodiments, the iodinated moiety of the multi-arm polymer is formed from an iodinated compound, and the reactive moiety is covalently linked to the hydrophilic polymer segment of the multi-arm polymer through an iodinated residue of the iodinated compound.

In some of these embodiments, the iodinated moiety is formed from an iodinated amino acid compound, and the reactive moiety is covalently linked to the hydrophilic polymer segment through a residue of the iodinated amino acid compound. In these embodiments the multi-arm polymers have polymer arms in which an iodinated amino acid compound residue is covalently linked to the hydrophilic polymer segment and in which a reactive moiety is covalently linked to the iodinated amino acid compound residue.

A few specific examples of iodinated amino acids for use in the present disclosure include the following: monoiodophenylalanine,

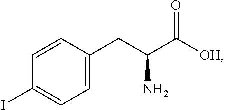 [SMILES: N[C@@H](Cc1ccc(I)cc1)C(=O)O]

monoiodotyrosine,

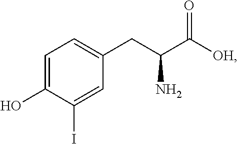 [SMILES: N[C@@H](Cc1ccc(O)c(I)c1)C(=O)O]

diiodotyrosine,

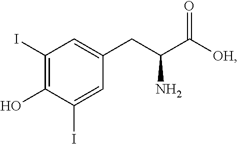 [SMILES: N[C@@H](Cc1cc(I)c(O)c(I)c1)C(=O)O]

diiodothyronine,

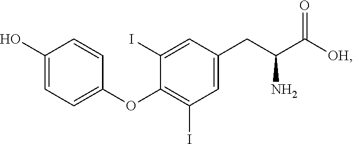 [SMILES: N[C@@H](Cc1cc(I)c(Oc2ccc(O)cc2)c(I)c1)C(=O)O]

triiodothyronine also known as T3,

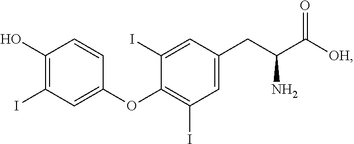 [SMILES: N[C@@H](Cc1cc(I)c(Oc2ccc(O)c(I)c2)c(I)c1)C(=O)O]

tetraiodothyronine also known as thyroxine or T4,

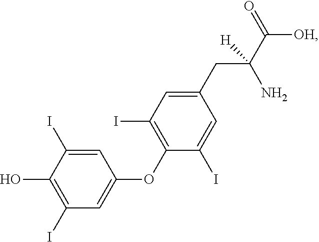 [SMILES: N[C@@H](Cc1cc(I)c(Oc2cc(I)c(O)c(I)c2)c(I)c1)C(=O)O]

and 6-iodo-L-DOPA.

In embodiments, a hydroxy-terminated multi-arm polymer, such as those described above, among others, may be reacted with a carboxylic acid group of an iodinated amino acid the presence of a suitable coupling agent (e.g., a carbodiimide coupling agent such as N,N′-dicyclohexylcarbodiimide (DCC), 1-ethyl-3-(3-dimethyl′propyl)carbodiimide (EDC), 1,3-diisopropylcarbodiimide (DIC), N-hydroxybenzotriazole (HOBt), BOP reagent, and/or another coupling agent) to form an ester linkage. To prevent the amino group(s) of the amino acid from reacting with the carboxylic acid group of the amino acid, the amino group(s) of the amino acid is protected using a suitable protective group. Examples of amine protective groups for use in conjunction with amide coupling reactions include tert-butoxycarbonyl (Boc) groups, carboxybenzyl (CBz) or (Z) groups, trifluoroacetyl (TFA) groups, and 9-fluorenylmethoxycarbonyl (Fmoc) groups, among others. Commercially available Boc-protected iodinated amino acids include Boc-thyroxine, Boc-iodotyrosine, Boc-iodophenylalanine, Boc-diiodophenylalanine, and Boc-triiodophenylalanine.

After amide coupling is complete, the resulting protected amino acid residue can be deprotected using known techniques, such as acid deprotection (e.g., HCl, trifluoroacetic acid, etc.) for Boc groups, hydrogenation (e.g., H₂/Pd—C) for CBz groups, base deprotection (e.g., with NaOH, KOH, etc.) for TFA groups, or amine deprotection (e.g., piperidine, piperazine, cyclohexylamine, ethanolamine, etc.) for Fmoc groups.

The deprotected amino group of the iodinated-amino-acid-terminated multi-arm polymer is then available for further reaction. In various embodiments, the deprotected amino group is reacted in a ring-opening reaction with an iodinated or non-iodinated cyclic anhydride, such as one of those described above, to form a carboxylic-acid-terminated multi-arm polymer wherein the carboxylic acid group is linked to the amino-acid residue through an amide-containing linkage.

A reactive moiety, for example, an electrophilic moiety, may then be linked to the carboxylic acid group. In some embodiments, an N-hydroxy cyclic imide compound (e.g., N-hydroxysuccinimide, N-hydroxymaleimide, N-hydroxyglutarimide, N-hydroxyphthalimide, or N-hydroxy-5-norbornene-2,3-dicarboxylic acid imide, also known as N-hydroxybicyclo[2.2.1]hept-5-ene-2,3-dicarboxylic acid imide (HONB), etc.) may be reacted with the carboxylic acid group, as described above, to form a reactive cyclic imide ester (e.g., a succinimide ester group, a maleimide ester group, a glutarimide ester group, a phthalimide ester group, a diglycolimide ester group, a bicyclo[2.2.1]hept-5-ene-2,3-dicarboxylic acid imide ester group, etc.) that is linked to the amino-acid residue through the amide linkage that was formed in the ring-opening reaction described above. In this way, a number of activated ester groups can be formed.

In a particular embodiment, a hydroxy-terminated multi-arm PEG,

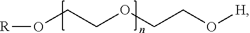

where R represent an iodinated core and the remaining polymer arms of the hydroxy-terminated multi-arm PEG, is reacted with an amine-protected iodinated amino acid, specifically, Boc-3,5-diiodotyrosine,

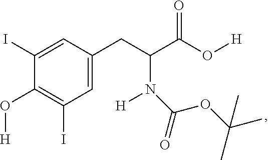 [SMILES: CC(C)(C)OC(=O)NC(Cc1cc(I)c(O)c(I)c1)C(=O)O]

in an ester coupling reaction to provide a multi-arm polymer that is terminated with amine-protected iodinated amino-acid residues, specifically, a Boc-protected diiodotyrosine-residue-terminated multi-arm polymer,

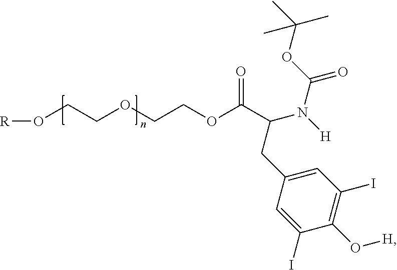

referred to herein as multi-arm-PEG-Boc-DIT. The ester coupling reaction may be conducted using a suitable coupling agent such as DCC, DIC, or EDC. The resulting ester group provides a hydrolysable linkage within the multi-arm polymer.

Then, the protective group of the amine-protected iodinated amino-acid-residue-terminated multi-arm polymer is removed. Specifically, the Boc protection group of the multi-arm-PEG-Boc-DIT, is removed using a suitable acid such as HCl or trifluoroacetic acid to obtain a multi-arm polymer having arms that are terminated with an amino-containing iodinated amino-acid residue, specifically, a diiodotyrosine-terminated multi-arm polymer, referred to herein as multi-arm-PEG-DIT,

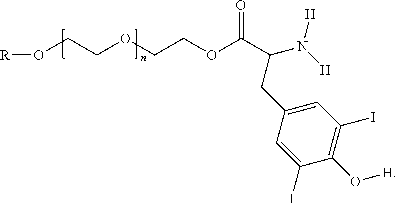

The amino groups of the iodinated amino-acid-terminated multi-arm polymer (i.e., the multi-arm-PEG-DIT) are then reacted in a ring opening reaction with a cyclic acid anhydride, specifically, glutaric anhydride, to form a carboxylic-acid-substituted polymer in which carboxylic acid groups are linked to the iodinated amino-acid residues through an amide linkage,

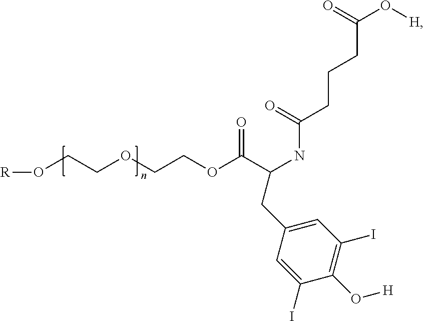

referred to herein as multi-arm-PEG-DIT-GA. Lastly, an N-hydroxy-cyclic-imide compound, specifically, N-hydroxysuccinimide, is coupled with the multi-arm-PEG-DIT-GA to produce a cyclic-imide-ester-substituted, iodinated-amino-acid-residue-terminated multi-arm polymer, specifically, a succinimidyl-glutaramide-substituted, 3,5-diiodotyrosine-residue-terminated multi-arm PEG,

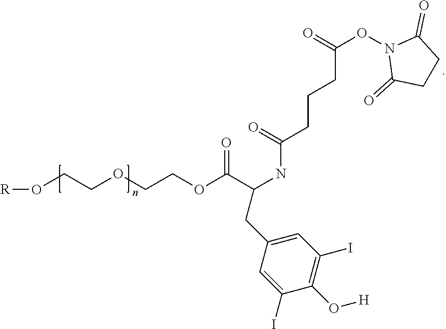

This strategy is widely applicable to other hydroxy-terminated polymers, including hydroxy-terminated multi-arm polymers having hydrophilic polymer segments besides PEG segments, such as those disclosed above.

Using these and other strategies, a variety of multi-arm polymers having a variety of iodinated polyol residue cores and a variety of cyclic-imide-ester-terminated polymer arms, in which the cyclic imide ester is attached to a variety of polymer segments through a variety of cyclic anhydride residues and a variety iodinated amino acid residues, may be provided.

With reference again to FIG. 1B, it can be seen that these multi-arm polymers fall into the category wherein the reactive moiety 106 and the functional/iodinated moiety 108 are positioned on one side of a hydrolysable ester group at an end of the polymer arm and the hydrophilic polymer segment 104 and the functional/iodinated core region 102 positioned on an opposite side of the hydrolysable ester group.

As noted above and as seen above in conjunction with FIGS. 1A-1B, multi-arm polymers in accordance with the present disclosure include those with polymer arms in which a reactive moiety is linked to a hydrophilic polymer segment through a linkage that comprises an ester group.

In some embodiments, the reactive moiety and iodinated moiety are covalently linked to the hydrophilic polymer segment through a residue of an amino alcohol. in some of these embodiments, each polymer arm comprises an amino-alcohol residue that is attached to the hydrophilic polymer segment through an ether group, a reactive moiety is attached to the amino-alcohol residue through a linkage that comprises a hydrolysable ester group, and a functional/iodinated moiety is attached the amino-alcohol residue through a linkage comprising an amide group.

Examples of amino-alcohol groups for used in the present disclosure include for example, C1-C6-alkyl groups that are substituted with an amino group and a hydroxyl group, which include, for example, hydroxyaminomethyl groups, hydroxyaminoethyl groups, hydroxyaminopropyl groups, hydroxyaminobutyl groups, hydroxyaminopentyl groups, or hydroxyaminohexyl groups. These C1-C6-hydroxyaminoalkyl groups are provided on polymer arms that comprise hydrophilic polymer segments having C1-C6-hydroxyaminoalkoxy end groups, including hydroxyaminomethyloxy end groups, hydroxyaminoethyloxy end groups, hydroxyaminopropyloxy end groups, hydroxyaminobutyloxy end groups, hydroxyaminopentyloxy end groups, or hydroxyaminohexyloxy end groups. In some embodiments, the amino-alcohol groups are vicinal amino-alcohol groups in which the amino group and the hydroxyl group are attached to adjacent carbon atoms.

In some embodiments, a hydroxy-terminated multi-arm polymer like that described above, for example, a hydroxy-terminated multi-arm polyethylene glycol (PEG),

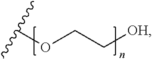

among others, is employed (only a single arm is shown). In a first step, the hydroxy-terminated multi-arm may be reacted with 1,1-dimethylethyl 2,2-dimethyl-5-[[[(4-methylphenyl)sulfonyl]oxy]methyl]-3-oxazolidinecarboxylate,

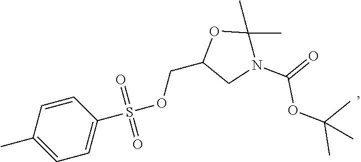 [SMILES: Cc1ccc(S(=O)(=O)OCC2CN(C(=O)OC(C)(C)C)C(C)(C)O2)cc1]

in a substitution reaction to form a multi-arm polymer in which the terminal hydroxyl group is replaced with a tert-butyl 2,2-dimethyloxazolidine-3-carboxylate group,

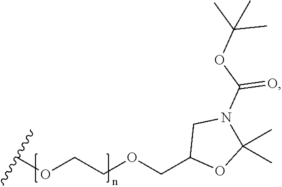

where n represents and integer and may have a value ranging, for example, from 5 to 500. In a subsequent step, the multi-arm polymer is treated with an acid such as hydrochloric acid or TFA in the presence of water to remove the protective groups, thereby providing an amino-alcohol-terminated multi-arm polymer in which an amino-alcohol end group is attached to the PEG segment of each polymer arm through an ether group, for example, a 2-hydroxy-3-amino-n-propoxy-terminated multi-arm PEG:

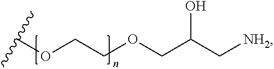

where n represents and integer and may have a value ranging, for example, from 5 to 500. It is noted that only a single arm of the multi-arm polymer is shown, and the remaining arms and the iodinated core are not illustrated.

The amino group of the amino-alcohol-terminated multi-arm polymer is subsequently used for the attachment of an iodinated moiety to the multi-arm polymer, and the hydroxyl group of the amino-alcohol-terminated multi-arm polymer is subsequently used for the attachment of a reactive moiety to the multi-arm polymer.

Turning first to the attachment of the iodinated moiety, in various embodiments, the amino group provided by the amino-alcohol group of the amino-alcohol-terminated multi-arm polymer may be reacted in an amide coupling reaction with a carboxylic-acid group of a carboxylic-acid-containing iodinated compound. The carboxylic-acid-containing iodinated compound may comprise at least one iodinated aromatic group (e.g., a monocyclic or multicyclic aromatic structure that is substituted with one, two, three, four, five, six or more iodine atoms) and a carboxylic acid group. In preferred embodiments, the amino group may be reacted with the carboxylic acid group in the presence of a suitable amide coupling agent. Examples of suitable amide coupling agents include carbodiimide coupling agents, such as N,N′-dicyclohexylcarbodiimide (DCC), 1-ethyl-3-(3-dimethyl′propyl)carbodiimide (EDC), and 1,3-diisopropylcarbodiimide (DIC), N-hydroxybenzotriazole (HOBt), BOP reagent, and TBTU (2-(1H-Benzotriazole-1-yl)-1,1,3,3-tetramethylaminium tetrafluoroborate), among others.

Carboxylic-acid-containing iodinated compounds for use in the present disclosure include triiodobenzoic acid

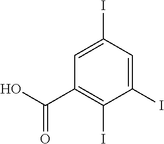 [SMILES: O=C(O)c1cc(I)cc(I)c1I]

(CAS #88-82-4), diatrizoic acid,

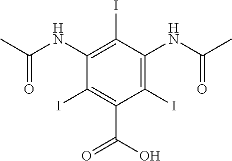 [SMILES: CC(=O)Nc1c(I)c(NC(C)=O)c(I)c(C(=O)O)c1I]

(CAS #117-96-4), N-acetyl-3,5-diiodo-L-tyrosine,

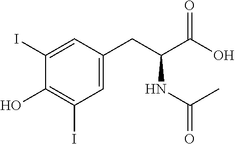 [SMILES: CC(=O)N[C@@H](Cc1cc(I)c(O)c(I)c1)C(=O)O]

(CAS #1027-28-7), N-acetyl-3-diiodo-L-tyrosine,

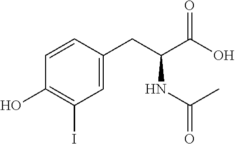 [SMILES: CC(=O)N[C@@H](Cc1ccc(O)c(I)c1)C(=O)O]

(CAS #1023-47-8), and N-acetyl-thyroxine,

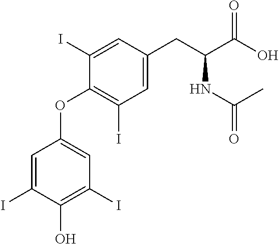 [SMILES: CC(=O)N[C@@H](Cc1cc(I)c(Oc2cc(I)c(O)c(I)c2)c(I)c1)C(=O)O]

(CAS #26041-51-0), among others.

Various additional carboxylic-acid-containing iodinated compounds, along with their CAS numbers, are listed in the following table:

| Name | CAS | # I atoms |
|---|---|---|
| 2-Iodobenzoic acid | 88-67-5 | 1 |
| 4-Iodobenzoic acid | 619-58-9 | 1 |
| 3-Iodobenzoic acid | 618-51-9 | 1 |
| 4-Iodopicolinic acid | 405939-79- | 1 |
| | 9 | |
| 3,5-Diiodobenzoic acid | 19094-48-5 | 2 |
| 3,4-Diiodobenzoic acid | 35674-20-5 | 2 |
| 3,5-Diiodo-4-(4-methoxyphenoxy)benzoic acid | 34043-77-1 | 2 |
| Diatrizoate | 117-96-4 | 3 |
| 2,3,5-Triiodobenzoic acid | 88-82-4 | 3 |
| Acetrizoic acid | 85-36-9 | 3 |
| Metrizoate | 1949-45-7 | 3 |
| 3,4,5-Triiodobenzoic acid | 2338-20-7 | 3 |
| Ioxitalamic acid | 28179-44-4 | 3 |
| Iothalamic acid | 2276-90-6 | 3 |
| 2,4,6-Triiodo-1,3,5-benzenetricarboxylic acid | 79211-41-9 | 3 |
| 2,4,6-Triiodobenzoic acid | 2012-31-9 | 3 |
| enzoic acid, 3-[[[2-Hydroxy-1- | 87932-11-4 | 3 |
| (hydroxymethyl)ethyl]amino]carbonyl]-5-[(2- | | |
| hydroxy-1-oxopropyl)amino]-2,4,6-triiodo-, (S)- | | |
| (9CI) | | |
| Ioseric acid | 51876-99-4 | 3 |
| 4-[4-(Acetyloxy)-3-iodophenoxy]-3,5- | 2260-084 | 3 |
| diiodobenzoic acid | | |
| Ioglicic acid | 49755-67-1 | 3 |
| 2,4,6-Triiodo-3-[(1-oxo-3,6,9,12,15- | 16024-67-2 | 3 |
| pentaoxahexadec-1-yl)amino]benzoic acid | | |
| 3-[[(1,1-Dimethylethoxy)carbonyl]amino]-2,4,6- | 2358047- | 3 |
| triiodobenzoic acid | 48-8 | |
| 2,3,4,6-Tetraiodobenzoic acid | 71463-71-3 | 4 |
| Tetraform | 2055-97-2 | 4 |
| 2,3,5,6-Tetraiodo-1,4-benzenedicarboxylic acid | 7606-84-0 | 4 |
| N-Acetyl-O-(4-hydroxy-3,5-diiodophenyl)-3,5- | 26041-51-0 | 4 |
| diiodo-L-tyrosine | | |
| D-Tyrosine, N-[(1,1-dimethylethoxy)carbonyl]- | 89624-64-6 | 4 |
| O-(4-hydroxy-3,5-diiodophenyl)-3,5-diiodo- | | |
| (9CI, ACI) | | |
| 2,3,4,5,6-Pentaiodobenzoic acid | 64385-02-0 | 5 |
| Ioxaglic acid | 59017-64-0 | 6 |
| Ioglycamic acid | 2618-25-9 | 6 |
| Iocarmic acid | 10397-75-8 | 6 |

Carboxylic-acid-containing iodinated compounds for use in the present disclosure can also be formed by reacting an amine-containing iodinated compound with a cyclic anhydride in a ring-opening reaction. The amine-containing iodinated compound may comprise, for example, an iodinated aromatic group and an amine group. The amine-containing iodinated compound can be used to form, for example, a carboxy-C1-C6-alkylamido-containing iodinated compound.

Various cyclic anhydrides are listed above. Some specific examples of amine-containing aromatic iodinated compounds are found in the table to follow.

| Name | CAS | # I atoms |
|---|---|---|
| 4-Iodobenzylamine | 39959-59-6 | 1 |
| 5-Amino-N,N′-bis(2,3-dihydroxypropyl)- | 76801-93-9 | 3 |
| 2,4,6-triiodoisophthalamide | | |
| 3,5-Diiodobenzenamine | 35122-96-4 | 2 |
| 2,4,6-Triiodobenzenamine | 24154-37-8 | 3 |

In some embodiments, the amine-containing aromatic iodinated species that is reacted with a cyclic anhydride in a ring-opening reaction is a C₁-C₅-alkyl ester of an iodinated amino acid, preferably a methyl ester of an iodinated amino acid. Specific examples of iodinated amino-acid C₁-C₅-alkyl esters include the following: monoiodo-phenylalanine methyl ester,

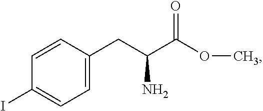 [SMILES: COC(=O)[C@@H](N)Cc1ccc(I)cc1]

monoiodotyrosine methyl ester,

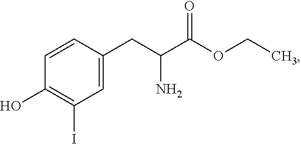 [SMILES: CCOC(=O)C(N)Cc1ccc(O)c(I)c1]

diiodotyrosine methyl ester,

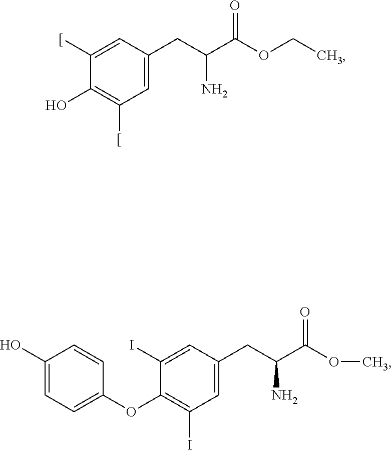

triiodothyronine methyl ester, also known as T3 methyl ester,

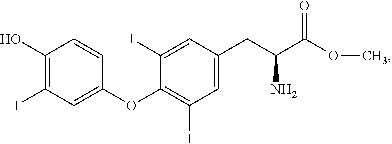 [SMILES: COC(=O)[C@@H](N)Cc1cc(I)c(Oc2ccc(O)c(I)c2)c(I)c1]

tetraiodothyronine methyl ester, also known as thyroxine methyl ester or T4 methyl ester,

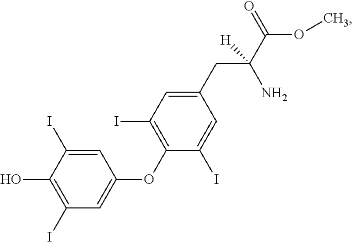 [SMILES: COC(=O)[C@@H](N)Cc1cc(I)c(Oc2cc(I)c(O)c(I)c2)c(I)c1]

thyroxine methyl ester,

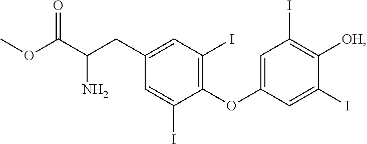 [SMILES: COC(=O)C(N)Cc1cc(I)c(Oc2cc(I)c(O)c(I)c2)c(I)c1]

and bis-thyroxine methyl ester (w/multi-arm-linker),

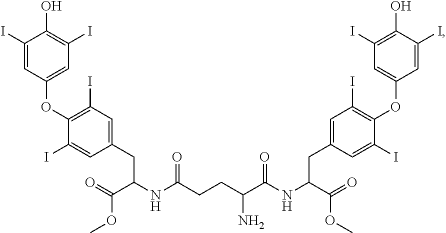 [SMILES: COC(=O)C(Cc1cc(I)c(Oc2cc(I)c(O)c(I)c2)c(I)c1)NC(=O)CCC(N)C(=O)NC(Cc1cc(I)c(Oc2cc(I)c(O)c(I)c2)c(I)c1)C(=O)OC]

and 6-iodo-L-DOPA methyl ester, among others.

In one specific embodiment, an iodinated amino-acid C₁-C₅-alkyl ester, specifically, diiodotyrosine methyl ester, is reacted in a ring-opening reaction with a cyclic anhydride, specifically, succinic anhydride, to form a carboxyalkylamido-modified iodinated amino-acid C₁-C₅-alkyl ester, specifically, 4-[[1-[(4-hydroxy-3,5-diiodo-phenyl)methyl]-2-methoxy-2-oxo-ethyl]amino]-4-oxo-butanoic acid,

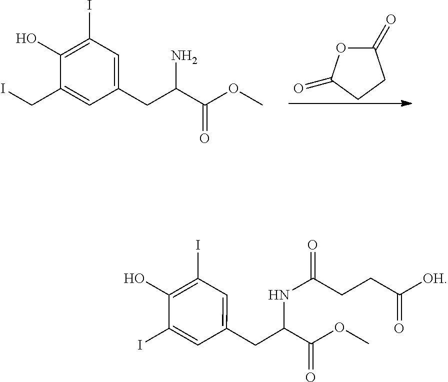

In various embodiments, the amino groups provided by an amino-alcohol-terminated multi-arm polymer, which comprises an iodinated core region and a plurality of polymer arms that each comprise an amino-alcohol end group that is attached through an ether group to a hydrophilic polymer segment, are reacted with a carboxylic-acid group of a carboxylic-acid-containing iodinated compound in the presence of a coupling agent to form a multi-arm polymer which comprises an iodinated core region and a plurality of polymer arms that each comprise a residue of the carboxylic-acid-containing iodinated molecule, which is attached to a residue of each amino-alcohol group of the amino-alcohol-terminated multi-arm polymer through a linkage comprising an amide group.

In a specific embodiment, the amino groups of the 2-hydroxy-3-amino-n-propoxy-terminated multi-arm PEG described above are reacted in an amide coupling reaction with diatrizoic acid,

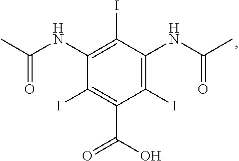 [SMILES: CC(=O)Nc1c(I)c(NC(C)=O)c(I)c(C(=O)O)c1I]

in the presence of a coupling agent, such as EDC, to form 2-hydroxy-3-(3,5-diacetamido-2,4,6-triiodobenzamido)-n-propoxy-terminated multi arm PEG,

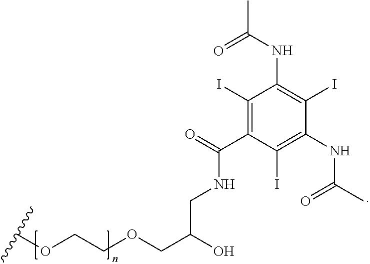

In another specific embodiment, the amino groups of the 2-hydroxy-3-amino-n-propoxy-terminated multi-arm PEG described above are reacted in an amide coupling reaction with a carboxyalkylamido-modified iodinated amino-acid C₁-C₅-alkyl ester, specifically, 4-[[1-[(4-hydroxy-3,5-diiodo-phenyl)methyl]-2-methoxy-2-oxo-ethyl]amino]-4-oxo-butanoic acid, described above, preferably in the presence of a coupling agent, to form 2-hydroxy-3-(4-[[1-[(4-hydroxy-3,5-diiodo-phenyl)methyl]-2-methoxy-2-oxo-ethyl]amino]-4-oxo-butanoic acid)-n-propoxy-terminated multi-arm PEG,

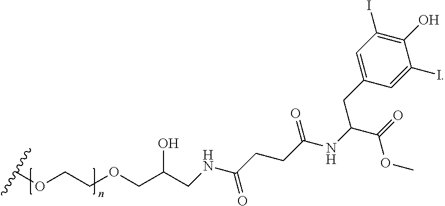

It is noted that the multi-arm PEG contains an iodinated amino-acid C₁-C₅-alkyl ester residue, specifically, a diiodotyrosine methyl ester residue, which is attached to an amino-alcohol residue through a linkage that contains two amide groups.

In various embodiments, the hydroxyl groups provided by amino-alcohol groups of the amino-alcohol-terminated multi-arm polymer, are then reacted with an iodinated or non-iodinated cyclic anhydride, such as one of those described elsewhere herein, to form a multi-arm polymer which comprises a plurality of polymer arms that each comprise a carboxylic acid group that is linked through an ester-containing linkage to a residue of the amino-alcohol group of the amino-alcohol-terminated multi-arm polymer. Then, the carboxylic acid groups provided by the ring-opening reaction of the cyclic anhydride are reacted in an amide coupling reaction with an N-hydroxy cyclic imide compound, such as one of those described elsewhere herein, to form cyclic-imidyl ester groups that are coupled through hydrolysable ester-containing linkages to residues of the amino-alcohol group.

In one particular example, the hydroxy groups of the 2-hydroxy-3-(3,5-diacetamido-2,4,6-triiodobenzamido)-n-propoxy-terminated multi-arm PEG described above are reacted in a ring-opening reaction with glutaric anhydride to form 2-(glutaric acid)-3-(3,5-diacetamido-2,4,6-triiodobenzamido)-n-propoxy-terminated multi-arm PEG, also referred to herein as 2-(carboxytrimethylenecarbonyloxy)-3-(3,5-diacetamido-2,4,6-triiodobenzamido)-n-propoxy-terminated multi-arm PEG,

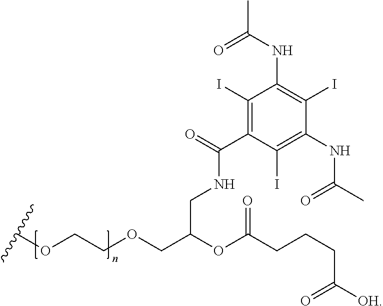

This compound is then reacted in an amide coupling reaction with N-hydroxy succinimide (typically in the presence of a coupling agent) to form 2-(succinimidyl glutarate)-3-(3,5-diacetamido-2,4,6-triiodobenzamido)-n-propoxy-terminated multi-arm PEG, also referred to as 2-(succinimidyloxycarbonyltrimethylenecarbonyloxy)-3-(3,5-diacetamido-2,4,6-triiodobenzamido)-n-propoxy-terminated multi-arm PEG,

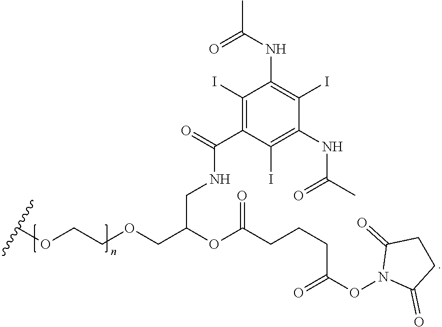

In another example, the hydroxy groups of the 2-hydroxy-3-(4-[[1-[(4-hydroxy-3,5-diiodo-phenyl)methyl]-2-methoxy-2-oxo-ethyl]amino]-4-oxo-butanoic acid)-n-propoxy-terminated multi-arm PEG described above may be reacted in a ring-opening reaction with glutaric anhydride to form 2-(glutaric acid)-3-(4-[[1-[(4-hydroxy-3,5-diiodo-phenyl)methyl]-2-methoxy-2-oxo-ethyl]amino]-4-oxo-butanoic acid)-n-propoxy-terminated multi-arm PEG,

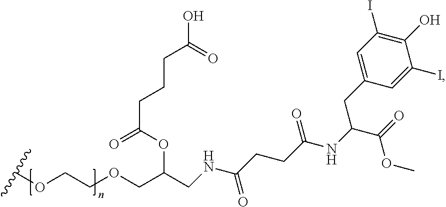

which is then reacted in an amide coupling reaction with N-hydroxy succinimide (typically in the presence of a coupling agent) to form 2-(succinimidyl glutarate)-3-(4-[[1-[(4-hydroxy-3,5-diiodo-phenyl)methyl]-2-methoxy-2-oxo-ethyl]amino]-4-oxo-butanoic acid)-n-propoxy-terminated multi-arm PEG,

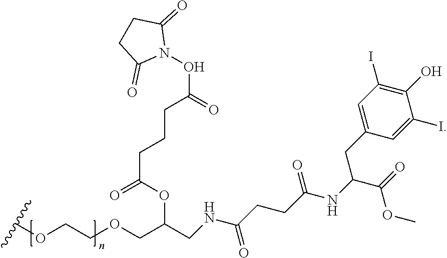

In some of these embodiments each polymer arm comprises an iodinated or non-iodinated carboxylic-acid-substituted cyclic anhydride compound residue that is attached to the hydrophilic polymer segment through an ester group, a reactive group is directly attached to the carboxylic-acid-substituted cyclic anhydride residue and a residue of a hydroxyl-containing iodinated molecule is attached the carboxylic-acid-substituted cyclic anhydride residue through a linkage that comprises two amide groups.

In some of these embodiments, a hydroxy-terminated multi-arm polymer, such as one of those described above, is reacted in a ring opening reaction with a carboxyl-protected carboxylic-acid-substituted cyclic anhydride compound to form a multi-arm polymer having multiple polymer arms that comprises an iodinated core region and a plurality of polymer arms that each comprise a carboxylic-acid-terminated hydrophilic polymer segment in which an end group that contains a protected carboxyl group and an unprotected carboxyl group is linked to a residue of the alcohol-terminated multi-arm polymer through an ester-containing linkage. Examples of carboxylic acid protecting groups include esters such as methyl esters, benzyl esters, tert-butyl esters, allyl esters, and trimethylsilylethyl esters.

Examples of carboxylic-acid-substituted cyclic anhydride compounds include mono-carboxylic-acid-substituted-cyclic anhydride compounds, di-carboxylic-acid-substituted-cyclic anhydride compounds, tri-carboxylic-acid-substituted-cyclic anhydride compounds, and tetra-carboxylic-acid-substituted-cyclic anhydride compounds, among others. Examples of carboxylic-acid-substituted cyclic anhydride compounds include -carboxylic-acid-substituted versions of the cyclic anhydride compounds described above. Particular examples include, for example, mono-carboxylic-acid-substituted-succinic anhydride, di-carboxylic-acid-substituted-succinic anhydride, tri-carboxylic-acid-substituted-succinic anhydride, tetra-carboxylic-acid-substituted-succinic anhydride, mono-carboxylic-acid-substituted-glutaric anhydride, di-carboxylic-acid-substituted-glutaric anhydride, tri-carboxylic-acid-substituted-glutaric anhydride, tetra-carboxylic-acid-substituted-glutaric anhydride, mono-carboxylic-acid-substituted-adipic anhydride, di-carboxylic-acid-substituted-adipic anhydride, tri-carboxylic-acid-substituted-adipic anhydride, tetra-carboxylic-acid-substituted-adipic anhydride, mono-carboxylic-acid-substituted-pimelic anhydride, di-carboxylic-acid-substituted-pimelic anhydride, tri-carboxylic-acid-substituted-pimelic anhydride, tetra-carboxylic-acid-substituted-pimelic anhydride, mono-carboxylic-acid-substituted-suberic anhydride, di-carboxylic-acid-substituted-suberic anhydride, tri-carboxylic-acid-substituted-suberic anhydride, and tetra-carboxylic-acid-substituted-suberic anhydride, among many others.

In one specific embodiment, a hydroxy-terminated multi-arm polymer, specifically, hydroxy-terminated multi-arm polyethylene glycol (PEG)

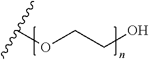

where n represents and integer and may have a value ranging, for example, from 5 to 500 (only a single arm of the multi-arm polymer is shown, and the remaining arms and the iodinated core are not illustrated) is reacted with a carboxyl-protected, carboxylic-acid-substituted cyclic anhydride compound, specifically, 1,1-dimethylethyl tetrahydro-2,6-dioxo-2H-pyran-4-carboxylate,

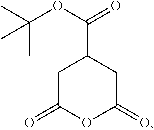

in a ring-opening reaction to form a multi-arm polymer,

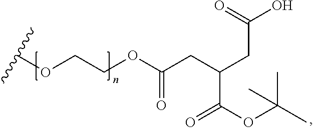

in which the terminal hydroxyl group is replaced with a 3-tert-butoxycarbonyl-5-methoxy-5-oxo-pentanoic acid group, specifically, a carboxy-trimethylene-carbonyloxy group in which the trimethylene group is substituted with a tert-butyl-protected-carboxy group.

Once a multi-arm polymer having multiple polymer arms that comprises an iodinated core region and a plurality of polymer arms that each comprise a carboxylic-acid-terminated hydrophilic polymer segment in which an end group that contains a protected carboxyl group and an unprotected carboxyl group is formed, the unprotected carboxylic-acid group is then available for further reaction in an amide coupling reaction with an amine-containing aromatic iodinated compound, which amide coupling reaction may be performed in the presence of a suitable coupling agent, such as those described above. Various amine-containing aromatic iodinated compounds are described above.

In some embodiments, the amine-containing aromatic iodinated compound is formed from a hydroxyalkyl-containing iodinated aromatic compound such as those described above, among others. The hydroxyalkyl-containing iodinated aromatic compound may comprise, for example, at least one iodinated aromatic group (e.g., a monocyclic or multicyclic aromatic structure that is substituted with one, two, three, four, five, six or more iodine atoms) and at least one hydroxyl group. In some embodiments, a hydroxyalkyl-containing iodinated aromatic compound is reacted with an isocyanate group of an amine-protected, isocyanate-containing amine compound, followed by deprotection of the amine group to form an amine-containing iodinated aromatic compound in which an amine group is coupled to an iodinated aromatic group through a urea-group-containing linkage. Examples of amine-protective groups for use in the present disclosure include tert-butoxycarbonyl (Boc) groups, carboxybenzyl (CBz) or (Z) groups, trifluoroacetyl (TFA) groups, and 9-fluorenylmethoxycarbonyl (Fmoc) groups, among others.

In one particular embodiment, a hydroxyalkyl-containing iodinated aromatic compound, specifically, acetal-protected iopamidol,

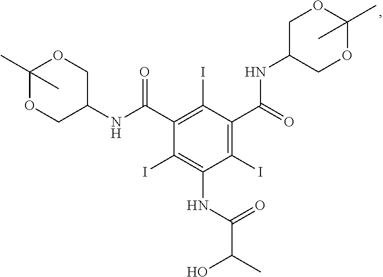

in which two vicinal-diol hydroxyl groups are protected and a non-vicinal-diol hydroxyl group remains unprotected, is reacted with an isocyanate group of an amine-protected, isocyanate-containing amine compound, specifically, 1,1-Dimethylethyl N-(3-isocyanatopropyl)carbamate,

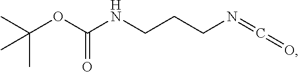

followed by deprotection of the acetal protection and the Boc protection using an acid such as HCl to form an amine-containing iodinated aromatic compound in which an amine group is coupled to an iodinated aromatic group through a urea-group-containing linkage, specifically, an amine-containing iodinated aromatic compound,

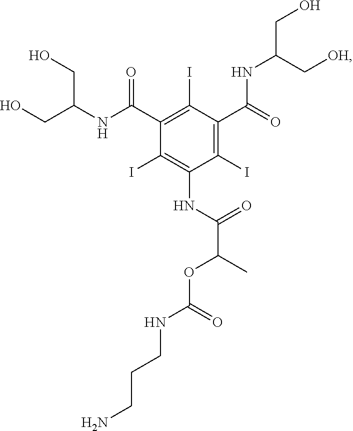 [SMILES: CC(OC(=O)NCCCN)C(=O)Nc1c(I)c(C(=O)NC(CO)CO)c(I)c(C(=O)NC(CO)CO)c1I]

in which an aminopropyl group is coupled to an iopamidol residue through a urea linkage.

Regardless of how the amine-containing iodinated aromatic compound is obtained, the amine-containing iodinated aromatic compound may be reacted in an amide coupling process with a multi-arm polymer having multiple polymer arms that comprises an iodinated core region and a plurality of polymer arms that each comprise a carboxylic-acid-terminated hydrophilic polymer segment in which an end group contains a protected carboxyl group and an unprotected carboxyl group as described above. Preferably the amide coupling is performed in the presence of a suitable coupling agent (e.g., a carbodiimide coupling agent, such as one of those described above). After the amide coupling is complete, the protection is removed from the protected carboxyl group, thereby providing an unprotected carboxyl group which is available for further reactions.

In a particular example, the amino group of the previously described amine-containing iodinated aromatic compound, in which an aminopropyl group is coupled to an iopamidol residue through a urea linkage, is reacted in an amide coupling step with the unprotected carboxyl group of the previously described a multi-arm polymer, which contains a protected-carboxy-substituted carboxyalkylcarbonyloxy group, specifically, a carboxy-trimethylene-carbonyloxy group in which the trimethylene group is substituted with a tert-butyl-protected-carboxy group. The amide coupling may be performed, for example, in the presence of EDC as a coupling agent. The tert-butyl ester protection is then removed, for example, in the presence of a base such as NaOH, thereby providing a multi-arm PEG,

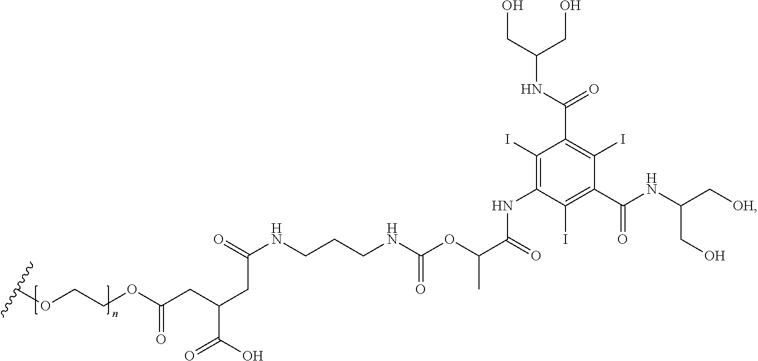

in which a carboxylic-acid-substituted cyclic anhydride residue, specifically, a 3-carboxy-glutaric anhydride residue, is attached to each PEG segment through a hydrolysable ester group and one or more hydroxyl-containing iodinated compound residues, specifically, an iopamidol residue, is attached to the carboxylic-acid-substituted cyclic anhydride residue through a urethane group and an amide group.

It can be seen that the carboxylic-acid-substituted cyclic anhydride compound residue contains a carboxyalkyl group, which may be further used to provide the multi-arm polymer with an electrophilic group. In some embodiments, an N-hydroxy cyclic imide compound (e.g., N-hydroxysuccinimide, N-hydroxymaleimide, N-hydroxyglutarimide, N-hydroxyphthalimide, or N-hydroxy-5-norbornene-2,3-dicarboxylic acid imide, also known as N-hydroxybicyclo[2.2.1]hept-5-ene-2,3-dicarboxylic acid imide (HONB), etc.) may be reacted with the carboxylic acid group of the carboxylic-acid-substituted cyclic anhydride compound residue in the presence of a suitable coupling agent (e.g., a carbodiimide coupling agent such as one of those described above) to form a reactive cyclic imide ester group (e.g., a succinimide ester group, a maleimide ester group, a glutarimide ester group, a phthalimide ester group, a diglycolimide ester group, a bicyclo[2.2.1]hept-5-ene-2,3-dicarboxylic acid imide ester group, etc.) that is linked to the carboxylic-acid-substituted cyclic anhydride compound residue.

In one particular embodiment, the above-described multi-arm PEG, may be reacted in an amide coupling reaction with N-hydroxy succinimide, in the presence of a coupling agent such as DCC, to form a multi-arm PEG,

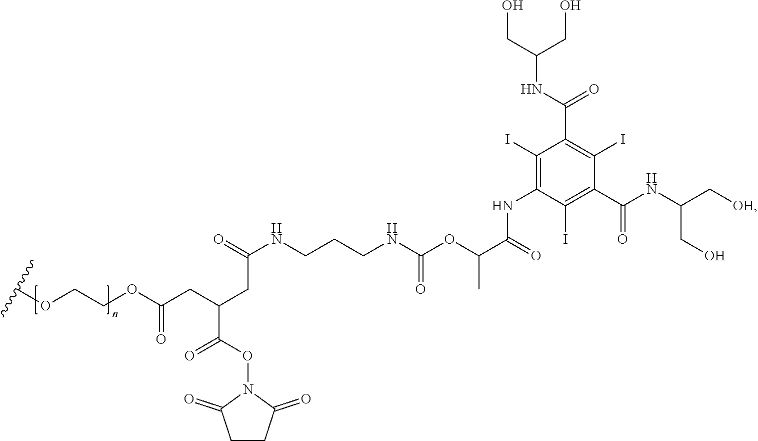

in which the polymer arms each comprise a hydrophilic polymer segment, specifically, a PEG segment, and a residue of a carboxylic-acid-substituted cyclic anhydride compound, specifically, a 3-carboxy-glutaric anhydride residue, that is attached to the PEG segment through a hydrolysable ester group, wherein a cyclic-imidyl group, specifically, a succinimidyl group, is attached to the 3-carboxy-glutaric anhydride residue through an ester group, and a residue of a hydroxyl-containing iodinated compound, specifically, an iopamidol residue, is attached to the 3-carboxy-glutaric anhydride residue through a urethane group and an amide group.

In other aspects, the present disclosure provides systems that comprise (a) a multi-arm polymer as described above, which comprises a functional core region and three or more polymer arms extending from the functional core region, each polymer arm of the three or more polymer arms comprising a hydrophilic polymer segment, a reactive moiety and a functional moiety, and (b) a multifunctional compound comprising a plurality of reactive groups that are reactive with the reactive moieties of the multi-arm polymer.

In some embodiments, the multifunctional compound is a compound having at two or more reactive groups, for example, having 2, 3, 4, 5, 6, 7, 8, 9, 10, 11, 12, 13, 14, 15, 16, 17, 18, 19, 20 or more second reactive groups. Suitable reactive groups include nucleophilic groups. Specific nucleophilic groups include amino groups and thiol groups.

Some specific examples of such multifunctional compounds include, for example, polyamines that contain two or more amino (—NH₂) groups (e.g., 2, 3, 4, 5, 6, 7, 8, 9, 10 or more amino groups in some embodiments), also referred to herein as polyamino compounds. Polyamino compounds suitable for use in the present disclosure include polyamino compounds that comprise two or more primary amine groups, for example, —(CH₂)ₓ—NH₂ groups where x is 1, 2, 3, 4, 5 or 6. Polyamino compounds suitable for use in the present disclosure include polyamino compounds that comprise two or more basic amino acid residues, including residues of amino acids having two or more primary amine groups, such as lysine and ornithine, for example, polyamines that comprise from 2 to 10 lysine and/or ornithine amino acid residues (e.g., dilysine, trilysine, tetralysine, pentalysine, diornithine, triornithine, tetraornithine, pentaornithine, etc.).

Further examples of polyamino compounds which may be used as the multifunctional compound include ethylenediamine, ethylenetriamine, diethylene triamine, hexamethylenetriiamine, di(heptamethylene) triamine, di(trimethylene) triamine, bis(hexamethylene) triamine, tris(2-aminoethyl)amine, tris(3-aminopropyl)amine, 1,3,5-tris-(2-aminoethyl)-[1,3,5]triazinane-2,4,6-trione, N,N,N′-tris(2-aminoethyl)ethylenediamine, triethylene tetramine, tripropylene tetramine, N,N′,N′-tetrakis(2-aminoethyl)-1,2-ethanediamine, tetraethylene pentamine, hexamethylene heptamine, pentaethylene hexamine, dimethyl octylamine, dimethyl decylamine, and JEFFAMINE polyetheramines available from Huntsman Corporation, and poly(allyl amine), among others.

Compounds having at least two amino groups may also be made from polyols such those described above. For example, hydroxyl groups of a polyol maybe reacted in an ester coupling reaction in the presence of a suitable coupling agent with a carboxyl group of a suitable amino acid compound in which the amino group of the amino acid is protected with a suitable protective group (e.g., a tert-butyloxycarbonyl (tBoc) protective group). Examples of amino acids may be selected, for example, from beta amino acids such as 3-aminopropanoic acid (also known as beta-alanine), gamma amino acids such as 4-aminobutanoic acid (also known as gamma-aminobutyric acid, or GABA), delta amino acids such as 5-aminopentanoic acid, epsilon amino acids such as 6-aminohexanoic acid, etc. Deprotection of the protective groups results in a compound in which a primary amine group is linked to a residue of the polyol through a hydrolysable ester group at the site of each of the hydroxyl groups of the polyol.

In some aspects of the present disclosure, crosslinked hydrogels are provided that comprise (a) a multi-arm polymer as described above, which comprises a functional core region and three or more polymer arms extending from the functional core region, each polymer arm of the three or more polymer arms comprising a hydrophilic polymer segment, a reactive moiety and a functional moiety and (b) a multifunctional compound comprising a plurality of reactive groups that are reactive with the reactive moieties of the multi-arm polymer.

As used herein, a “hydrogel” is a crosslinked polymer that contains water or can absorb water but does not dissolve when placed in water.

Although the present disclosure provides specific examples where the reactive moieties of the multi-arm polymer comprise electrophilic groups and the reactive groups of the multifunctional compound comprise nucleophilic groups, other pairs of reactive groups are also contemplated including the following, among others: (a) reactive moieties that comprise nucleophilic groups and reactive groups that comprise electrophilic groups, (b) reactive moieties that comprise strained alkyne groups and reactive groups that comprise azide groups, (c) reactive moieties that comprise azide groups and reactive groups that comprise strained alkyne groups, (d) reactive moieties that comprise strained alkene groups and reactive groups that comprise tetrazine groups, or (e) reactive moieties that comprise tetrazine groups and reactive groups that comprise strained alkene groups.

In various embodiments (e.g., where the functional core region is an iodinated core region and where the functional moieties are iodinated moieties), the hydrogels of the present disclosure are visible under fluoroscopy. In various embodiments, such crosslinked products have a radiopacity that is greater than 100 Hounsfield units (HU), beneficially anywhere ranging from 100 HU to 250 HU to 500 HU to 750 HU to 1000 HU to 2000 HU or more (in other words, ranging between any two of the preceding numerical values) for example, when measured on a bench-top micro CT system such as XtremeCT from Scanco Medical (Wangen-Brüttisellen, Switzerland) or similar.

The hydrogels of the present disclosure can be used in a variety of biomedical applications, including implants, medical devices, and pharmaceutical compositions.

In some aspects of the present disclosure, a system is provided that comprises (a) a first composition that comprises a multi-arm polymer as described herein and (b) a second composition that comprises multifunctional compound as described herein, wherein when the first and second compositions are combined, covalent crosslinks form between the multi-arm polymer and the multifunctional compound, creating a hydrogel.

The first composition may be a first fluid composition comprising the multifunctional compound or a first dry composition that comprises the multifunctional compound, to which a suitable fluid such as water for injection, saline, etc. can be added to form a first fluid composition. In addition to the multifunctional compound, the first composition may further comprise additional agents, including therapeutic agents, imaging agents, colorants, tonicity adjusting agents, suspension agents, wetting agents, and pH adjusting agents as described below.

The second composition may be a second fluid composition comprising the multi-arm polymer or a second dry composition that comprises the multi-arm polymer, to which a suitable fluid such as water for injection, saline, etc. can be added to form a second fluid composition. In addition to the multi-arm polymer, the second composition may further comprise additional agents, including therapeutic agents, imaging agents, colorants, tonicity adjusting agents, suspension agents, wetting agents, and pH adjusting agents as described below.

In some embodiments, the system is configured to combine a first fluid composition comprising the multifunctional compound with a second fluid comprising the multi-arm polymer, and the first composition, the second composition, or both, may optionally comprise additional agents as described herein. Upon mixing the first and second fluid compositions, the multifunctional compound crosslink with the multi-arm polymer, forming a hydrogel. The first and second fluid compositions may be combined to form crosslinked hydrogels, either in vivo or ex vivo.

In some embodiments, the multifunctional compound is initially combined with the multi-arm polymer under conditions where crosslinking between the multi-arm polymer and the multifunctional compound is suppressed (e.g., an acidic pH, in some embodiments). Then, when crosslinking is desired, the conditions are changed such that crosslinking is increased (e.g., a change from an acidic pH to a basic pH, in some embodiments), leading to crosslinking between the multifunctional compound and the multi-arm polymer, thereby forming a crosslinked product.

In some embodiments, the system comprises (a) a first composition that comprises a multifunctional compound as described herein and a multi-arm polymer as described herein and (b) a second composition, specifically, an accelerant composition, that contains an accelerant that is configured to accelerate a crosslinking reaction between the multifunctional compound and the multi-arm polymer. In some embodiments, the first composition may further comprise additional agents as described herein. In some embodiments, the second composition may further comprise additional agents as described herein.

In some embodiments, the system comprises (a) a first composition that comprises multifunctional compound as described herein, (b) a second composition that comprises a multi-arm polymer as described herein, and (c) a third composition, specifically, an accelerant composition, that contains an accelerant that is configured to accelerate a crosslinking reaction between the multifunctional compound and the multi-arm polymer. In some embodiments, the first composition may further comprise additional agents as described herein. In some embodiments, the second composition may further comprise additional agents as described herein. In some embodiments, the third composition may further comprise additional agents as described herein.

The first composition may be a first fluid composition comprising the multifunctional compound that is buffered to an acidic pH or a first dry composition that comprises the multifunctional compound, to which a suitable fluid such as water for injection, saline, an acidic buffer solution, etc. can be added to form a first fluid composition comprising the multifunctional compound that is buffered to an acidic pH. In some embodiments, for example, the acidic buffering composition may comprise monobasic sodium phosphate, among other possibilities. The first fluid composition comprising the multifunctional compound may have a pH ranging, for example, from about 3 to about 5. In addition to the multifunctional compound, the first composition may further comprise additional agents, including therapeutic agents, imaging agents, colorants, tonicity adjusting agents, suspension agents, wetting agents, and pH adjusting agents as described below.

The second composition may be a second fluid composition comprising the multi-arm polymer or a second dry composition that comprises the multi-arm polymer from which a fluid composition is formed, for example, by the addition of a suitable fluid such as water for injection, saline, or the first fluid composition comprising the multifunctional compound that is buffered to an acidic pH. In addition to the multi-arm polymer, the second composition may further comprise additional agents, including therapeutic agents, imaging agents, colorants, tonicity adjusting agents, suspension agents, wetting agents, and pH adjusting agents as described below.

In a particular embodiment, the first composition is a first fluid composition comprising the multifunctional compound (as well as additional agents in some cases) that is buffered to an acidic pH and the second composition is a dry composition that comprises the multi-arm polymer (as well as additional agents in some cases). The first composition may then be mixed with the second composition to provide a prepared fluid composition that is buffered to an acidic pH and comprises the multifunctional compound and the multi-arm polymer. In a particular example, a syringe may be provided that contains the first fluid composition comprising the multifunctional compound that is buffered to an acidic pH, and a vial may be provided that comprises the second dry composition (e.g., a powder) that comprises the multi-arm polymer. The syringe may then be used to inject the first fluid composition into the vial containing the multi-arm polymer to form a prepared fluid composition that is buffered to an acidic pH and contains the multifunctional compound and the multi-arm polymer, which can be withdrawn back into the syringe for administration.

The accelerant composition may be a fluid accelerant composition that is buffered to a basic pH or a dry composition that comprise a basic buffering composition to which a suitable fluid such as water for injection, saline, etc. can be added to form a fluid accelerant composition that is buffered to a basic pH. For example, the basic buffering composition may comprise sodium borate and dibasic sodium phosphate, among other possibilities. The fluid accelerant composition may have, for example, a pH ranging from about 9 to about 11. In addition to the above, the fluid accelerant composition may further comprise additional agents, including those described below.

A prepared fluid composition that is buffered to an acidic pH and comprises the multifunctional compound and the multi-arm polymer as described above (as well as additional agents in some cases) and a fluid accelerant composition that is buffered to basic pH as described above (which may include additional agents in some cases) may be combined form crosslinked hydrogels, either in vivo or ex vivo.

Additional agents for use in the compositions described herein include therapeutic agents, imaging agents, colorants, tonicity adjusting agents, suspension agents, wetting agents, and pH adjusting agents.

Examples of therapeutic agents include antithrombotic agents, anticoagulant agents, antiplatelet agents, thrombolytic agents, antiproliferative agents, anti-inflammatory agents, hyperplasia inhibiting agents, anti-restenosis agent, smooth muscle cell inhibitors, antibiotics, antimicrobials, analgesics, anesthetics, growth factors, growth factor inhibitors, cell adhesion inhibitors, cell adhesion promoters, anti-angiogenic agents, cytotoxic agents, chemotherapeutic agents, checkpoint inhibitors, immune modulatory cytokines, T-cell agonists, STING (stimulator of interferon genes) agonists, antimetabolites, alkylating agents, microtubule inhibitors, hormones, hormone antagonists, monoclonal antibodies, antimitotics, immunosuppressive agents, tyrosine and serine/threonine kinases, proteasome inhibitors, matrix metalloproteinase inhibitors, Bcl-2 inhibitors, DNA alkylating agents, spindle poisons, poly (DP-ribose)polymerase (PARP) inhibitors, and combinations thereof.

Examples of imaging agents include (a) fluorescent dyes such as fluorescein, indocyanine green, or fluorescent proteins (e.g. green, blue, cyan fluorescent proteins), (b) contrast agents for use in conjunction with magnetic resonance imaging (MRI), including contrast agents that contain elements that form paramagnetic ions, such as Gd(III), Mn(II), Fe(III) and compounds (including chelates) containing the same, such as gadolinium ion chelated with diethylenetriaminepentaacetic acid, (c) contrast agents for use in conjunction with ultrasound imaging, including organic and inorganic echogenic particles (i.e., particles that result in an increase in the reflected ultrasonic energy) or organic and inorganic echolucent particles (i.e., particles that result in a decrease in the reflected ultrasonic energy), (d) contrast agents for use in connection with near-infrared (NIR) imaging, which can be selected to impart near-infrared fluorescence to the hydrogels of the present disclosure, allowing for deep tissue imaging and device marking, for instance, NIR-sensitive nanoparticles such as carbon nanotubes (e.g., nanotubes derivatized with hydroxy or carboxyl groups, for instance, partially oxidized carbon nanotubes), dye-containing nanoparticles, such as dye-doped nanofibers and dye-encapsulating nanoparticles, and semiconductor quantum dots, among others, and NIR-sensitive dyes such as cyanine dyes, squaraines, phthalocyanines, porphyrin derivatives and boron dipyrromethane (BODIPY) analogs, among others, and (e) imageable radioisotopes including 99mTc, 201Th, 51Cr, 67Ga, 68Ga, 111In, 64Cu, 89Zr, 59Fe, 42K, 82Rb, 24Na, 45Ti, 44Sc, 51Cr and 177Lu, among others.

Examples of colorants include brilliant blue (e.g., Brilliant Blue FCF, also known as FD&C Blue 1), indigo carmine (also known as FD&C Blue 2), indigo carmine lake, FD&C Blue 1 lake, and methylene blue (also known as methylthioninium chloride), among others.

Examples of additional agents further include tonicity adjusting agents such as sugars (e.g., dextrose, lactose, etc.), polyhydric alcohols (e.g., glycerol, propylene glycol, mannitol, sorbitol, etc.) and inorganic salts (e.g., potassium chloride, sodium chloride, etc.), among others, suspension agents including various surfactants, wetting agents, and polymers (e.g., albumen, PEO, polyvinyl alcohol, block polymers, etc.), among others, and pH adjusting agents including various buffer solutes.

In various embodiments, a system is provided that includes one or more delivery devices for delivering first and second compositions to a subject.

In some embodiments, the system may include a delivery device that comprises a first reservoir that contains a first fluid composition that comprises multifunctional compound as described above and a second reservoir that contains a second fluid composition that comprises a multi-arm polymer as described above, wherein the first and second fluid compositions form a crosslinked product upon mixing. As previously noted, the first fluid composition, the second fluid composition, or both, may optionally comprise additional agents as described herein.

In some embodiments, the system may include a delivery device that comprises a first reservoir that contains a first fluid composition that comprises the multifunctional compound and the multi-arm polymer and is buffered to an acidic pH, such as the prepared fluid composition previously described, and a second reservoir that contains second fluid composition, such as the fluid accelerant composition previously described. As previously noted, the first fluid composition, the second fluid composition, or both, may optionally comprise additional agents as described herein.

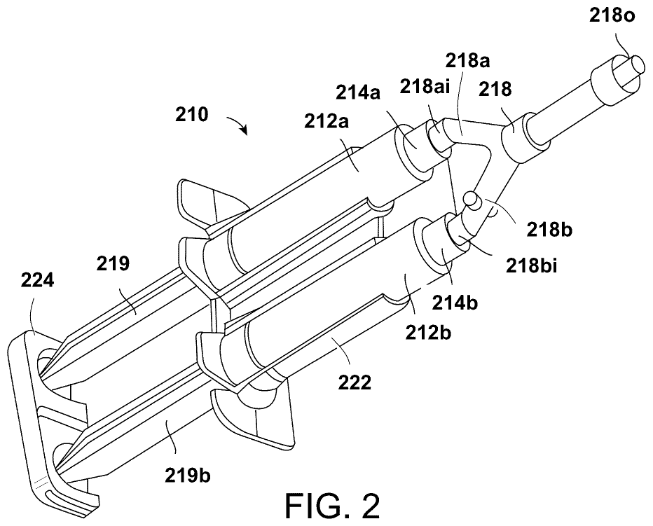

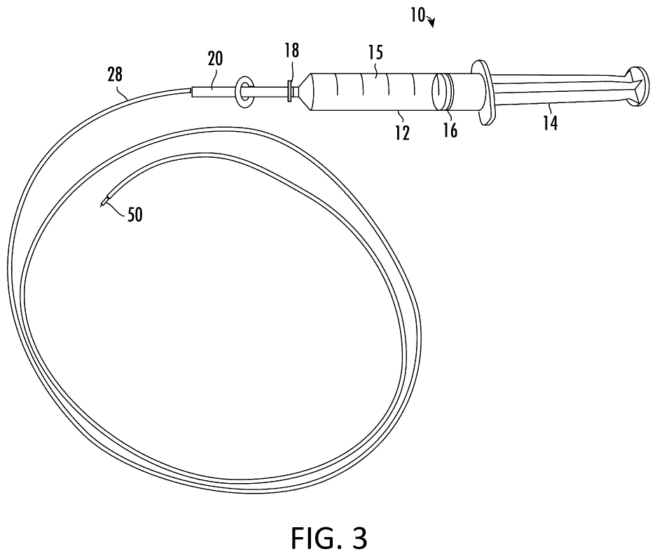

In either case, during operation, the first fluid composition and second fluid composition are dispensed from the first and second reservoirs and combined, whereupon the multifunctional compound and the multi-arm polymer and crosslink with one another to form a crosslinked hydrogel. In particular embodiments, and with reference to FIG. 2, the system may include a delivery device 210 that comprises a double-barrel syringe, which includes a first barrel 212a having a first barrel outlet 214a, which first barrel contains a first fluid composition as described above, a first plunger 219a that is movable in the first barrel 212a, a second barrel 212b having a second barrel outlet 214b, which second barrel 212b contains a second fluid composition as described above, and a second plunger 219b that is movable in the second barrel 212b. In some embodiments, the device 210 may further comprise a mixing section 218 having a first mixing section inlet 218ai in fluid communication with the first barrel outlet 214a, a second mixing section inlet 218bi in fluid communication with the second barrel outlet 214b, and a mixing section outlet 218o. Also shown are a syringe holder 222 configured to hold the first and second syringe barrels 212a, 212b, in a fixed relationship and a plunger cap 224 configured to hold the first and second plungers 219a, 219b in a fixed relationship. In some embodiments, the delivery device may further comprise a needle or catheter tube that is configured to receive the first and second fluid compositions from the first and second barrels. For example, a needle or catheter tube may be configured to form a fluid connection with an outlet of a mixing section by attaching the cannula or catheter tube to an outlet of the mixing section, for example, via a suitable fluid connector such as a luer connector.

In some embodiments, the delivery device may further comprise a needle or catheter tube that is configured to receive the first and second fluid compositions from the first and second barrels. For example, a needle or catheter tube may be configured to form a fluid connection with an outlet of a mixing section by attaching the cannula or catheter tube to an outlet of the mixing section, for example, via a suitable fluid connector such as a Luer connector.

As another example, the catheter may be a multi-lumen catheter that comprises a first lumen and a second lumen, a proximal end of the first lumen configured to form a fluid connection with the first barrel outlet and a proximal end of the second lumen configured to form a fluid connection with the second barrel outlet. In some embodiments, the multi-lumen catheter may comprise a mixing section having a first mixing section inlet in fluid communication with a distal end of the first lumen, a second mixing section inlet in fluid communication with a distal end of the second lumen, and a mixing section outlet.

During operation, when the first and second plungers are depressed, the first and second fluid compositions are dispensed from the first and second barrels, whereupon the first and second fluid compositions mix and ultimately crosslink to form a crosslinked hydrogel, which is administered onto or into tissue of a subject. For example, the first and second fluid compositions may pass from the first and second barrels, into the mixing section via first and second mixing section inlets, whereupon the first and second fluid compositions are mixed to form an admixture, which admixture exits the mixing section via the mixing section outlet. In some embodiments, a cannula or catheter tube is attached to the mixing section outlet, allowing the admixture to be administered to a subject after passing through the cannula or catheter tube.

As another example, the first fluid composition may pass from the first barrel outlet into a first lumen of a multi-lumen catheter and the second fluid composition may pass from the second barrel outlet into a second lumen of the multi-lumen catheter. In some embodiments the first and second fluid compositions may pass from the first and second lumen into a mixing section at a distal end of the multi-lumen catheter via first and second mixing section inlets, respectively, whereupon the first and second fluid compositions are mixed in the mixing section to form an admixture, which admixture exits the mixing section via the mixing section outlet.

Regardless of the type of device that is used to mix the first and second fluid compositions or how the first and second fluid compositions are mixed, immediately after an admixture of the first and second fluid compositions is formed, the admixture is initially in a fluid state and can be administered to a subject (e.g., a mammal, particularly, a human) by a variety of techniques. Alternatively, the first and second fluid compositions may be administered to a subject independently and a fluid admixture of the first and second fluid compositions formed in or on the subject. In either approach, a fluid admixture of the first and second fluid compositions is formed and used for various medical procedures.

For example, the first and second fluid compositions or a fluid admixture thereof can be injected to provide spacing between tissues, the first and second fluid compositions or a fluid admixture thereof can be injected (e.g., in the form of blebs) to provide fiducial markers or organ marking, the first and second fluid compositions or a fluid admixture thereof can be injected for tissue augmentation or regeneration, including cosmetic tissue augmentation, the first and second fluid compositions or a fluid admixture thereof can be injected as a filler or replacement for soft tissue, the first and second fluid compositions or a fluid admixture thereof can be injected to provide mechanical support for compromised tissue, the first and second fluid compositions or a fluid admixture thereof can be injected as a scaffold, the first and second fluid compositions or a fluid admixture thereof can be injected as an embolic composition, the first and second fluid compositions or a fluid admixture thereof can be injected for seminal vesicle occlusion, the first and second fluid compositions or a fluid admixture thereof can be injected as lifting agents for internal cyst removal, and/or the first and second fluid compositions or a fluid admixture thereof can be injected as a carrier of therapeutic agents in the treatment of diseases and cancers and the repair and regeneration of tissue, among other uses. The first and second fluid compositions or a fluid admixture thereof can also be injected into a left atrial appendage during a left atrial appendage closure procedure or injected for closure of an atrial septal defect. In some embodiments, the first and second fluid compositions or a fluid admixture thereof may be injected into the left atrial appendage after the introduction of a closure device such as the Watchman® left atrial appendage closure device available from Boston Scientific Corporation.

After administration of the compositions of the present disclosure (either separately as first and second fluid compositions that mix in vivo or as a fluid admixture of the first and second fluid compositions) a crosslinked hydrogel is ultimately formed at the administration location.

During and/or after administration, the compositions of the present disclosure can be imaged using a suitable imaging technique. Typically, the imaging technique is an x-ray-based imaging technique, such as computerized tomography or X-ray fluoroscopy, or a near near-IR fluorescence spectrometry-based technique.

As seen from the above, the compositions of the present disclosure may be used in a variety of medical procedures, including the following, among others: a procedure to implant a fiducial marker comprising a crosslinked product of the first and second fluid compositions, a procedure to implant a tissue regeneration scaffold comprising a crosslinked product of the first and second fluid compositions, a procedure to implant a tissue support comprising a crosslinked product of the first and second fluid compositions, a procedure to implant a tissue bulking agent comprising a crosslinked product of the first and second fluid compositions, a procedure to implant an embolic composition comprising a crosslinked product of the first and second fluid compositions, a procedure to implant a composition comprising a crosslinked product of the first and second fluid compositions to provide seminal vessel occlusion, a procedure to implant a lifting agent comprising a crosslinked product of the first and second fluid compositions, a procedure to introduce a left atrial appendage closure composition comprising a crosslinked product of the first and second fluid compositions, a procedure to implant a therapeutic-agent-containing depot comprising a crosslinked product of the first and second fluid compositions, a tissue augmentation procedure comprising implanting a crosslinked product of the first and second fluid compositions, a procedure to introduce a crosslinked product of the first and second fluid compositions between a first tissue and a second tissue to space the first tissue from the second tissue.

The first and second fluid compositions, fluid admixtures of the first and second fluid compositions, or the crosslinked products of the first and second fluid compositions may be injected in conjunction with a variety of medical procedures including the following: injection between the prostate or vagina and the rectum for spacing in radiation therapy for rectal cancer, injection between the rectum and the prostate for spacing in radiation therapy for prostate cancer, subcutaneous injection for palliative treatment of prostate cancer, transurethral or submucosal injection for female stress urinary incontinence, intra-vesical injection for urinary incontinence, uterine cavity injection for Asherman's syndrome, submucosal injection for anal incontinence, percutaneous injection for heart failure, intra-myocardial injection for heart failure and dilated cardiomyopathy, injection for closure of an atrial septal defect, injection for seminal vessel occlusion, trans-endocardial injection for myocardial infarction, intra-articular injection for osteoarthritis, spinal injection for spinal fusion, and spine, oral-maxillofacial and orthopedic trauma surgeries, spinal injection for posterolateral lumbar spinal fusion, intradiscal injection for degenerative disc disease, injection between pancreas and duodenum for imaging of pancreatic adenocarcinoma, resection bed injection for imaging of oropharyngeal cancer, injection around circumference of tumor bed for imaging of bladder carcinoma, submucosal injection for gastroenterological tumor and polyps, visceral pleura injection for lung biopsy, kidney injection for type 2 diabetes and chronic kidney disease, renal cortex injection for chronic kidney disease from congenital anomalies of kidney and urinary tract, intravitreal injection for neovascular age-related macular degeneration, intra-tympanic injection for sensorineural hearing loss, dermis injection for correction of wrinkles, creases and folds, signs of facial fat loss, volume loss, shallow to deep contour deficiencies, correction of depressed cutaneous scars, perioral rhytids, lip augmentation, facial lipoatrophy, stimulation of natural collagen production.

Where formed ex vivo, crosslinked hydrogels may be in any desired form, including a slab, a cylinder, a coating, or a particle. In some embodiments, the crosslinked hydrogel is dried and then granulated into particles of suitable size. Granulating may be by any suitable process, for instance by grinding (including cryogrinding), homogenization, crushing, milling, pounding, or the like. Sieving or other known techniques can be used to classify and fractionate the particles. crosslinked hydrogel particles formed using the above and other techniques may varying widely in size, for example, having an average size ranging from 50 to 950 microns.

In addition to a crosslinked hydrogel as described above, crosslinked hydrogel compositions in accordance with the present disclosure may contain additional agents, including therapeutic agents, imaging agents, colorants, tonicity adjusting agents, suspension agents, wetting agents, and pH adjusting agents as described above.

In various embodiments, kits are provided that include one or more delivery devices for delivering the crosslinked hydrogel to a subject. Such systems may include one or more of the following: a syringe barrel, which may or may not contain a crosslinked hydrogel as described herein; a vial, which may or may not contain a crosslinked hydrogel as described here; a needle; a flexible tube (e.g., adapted to fluidly connect the needle to the syringe); and an injectable liquid such as water for injection, normal saline or phosphate buffered saline. Whether supplied in a syringe, vial, or other reservoir, the crosslinked hydrogel may be provided in dry form (e.g., powder form) or in a form that is ready for injection, such as an injectable hydrogel form (e.g., a suspension of crosslinked hydrogel particles).

FIG. 14 illustrates a syringe 10 providing a reservoir for a hydrogel composition in accordance with the present disclosure. The syringe 10 may comprise a barrel 12, a plunger 14, and one or more stoppers 16. The barrel 12 may include a Luer adapter (or other suitable adapter/connector), e.g., at the distal end 18 of the barrel 12, for attachment to an injection needle 50 via a flexible catheter 29. The proximal end of the catheter 29 may include a suitable connection 20 for receiving the barrel 12. In other examples, the barrel 12 may be directly coupled to the injection needle 50. The syringe barrel 12 may serve as a reservoir, containing a hydrogel composition 15 for injection through the needle 50.

The crosslinked hydrogel compositions described herein can be used for a number of purposes.

For example, crosslinked hydrogel compositions can be injected to provide spacing between tissues, crosslinked hydrogel compositions can be injected (e.g., in the form of blebs) to provide fiducial markers, crosslinked hydrogel compositions can be injected for tissue augmentation or regeneration, crosslinked hydrogel compositions can be injected as a filler or replacement for soft tissue, crosslinked hydrogel compositions can be injected to provide mechanical support for compromised tissue, crosslinked hydrogel compositions be injected as a scaffold, and/or crosslinked hydrogel compositions can be injected as a carrier of therapeutic agents in the treatment of diseases and cancers and the repair and regeneration of tissue, among other uses.

During and/or after administration, the crosslinked hydrogel compositions of the present disclosure can be imaged using a suitable imaging technique.

As seen from the above, the crosslinked hydrogel compositions of the present disclosure may be used in a variety of medical procedures, including the following, among others: a procedure to implant a fiducial marker comprising a crosslinked hydrogel, a procedure to implant a tissue regeneration scaffold comprising a crosslinked hydrogel, a procedure to implant a tissue support comprising a crosslinked hydrogel, a procedure to implant a tissue bulking agent comprising a crosslinked hydrogel, a procedure to implant a therapeutic-agent-containing depot comprising a crosslinked hydrogel, a tissue augmentation procedure comprising implanting a crosslinked hydrogel, a procedure to introduce a crosslinked hydrogel between a first tissue and a second tissue to space the first tissue from the second tissue.

The crosslinked hydrogel compositions may be injected in conjunction with a variety of medical procedures including the following: injection between the prostate or vagina and the rectum for spacing in radiation therapy for rectal cancer, injection between the rectum and the prostate for spacing in radiation therapy for prostate cancer, subcutaneous injection for palliative treatment of prostate cancer, transurethral or submucosal injection for female stress urinary incontinence, intra-vesical injection for urinary incontinence, uterine cavity injection for Asherman's syndrome, submucosal injection for anal incontinence, percutaneous injection for heart failure, intra-myocardial injection for heart failure and dilated cardiomyopathy, trans-endocardial injection for myocardial infarction, intra-articular injection for osteoarthritis, spinal injection for spinal fusion, and spine, oral-maxillofacial and orthopedic trauma surgeries, spinal injection for posterolateral lumbar spinal fusion, intradiscal injection for degenerative disc disease, injection between pancreas and duodenum for imaging of pancreatic adenocarcinoma, resection bed injection for imaging of oropharyngeal cancer, injection around circumference of tumor bed for imaging of bladder carcinoma, submucosal injection for gastroenterological tumor and polyps, visceral pleura injection for lung biopsy, kidney injection for type 2 diabetes and chronic kidney disease, renal cortex injection for chronic kidney disease from congenital anomalies of kidney and urinary tract, injection for seminal vessel occlusion, intravitreal injection for neovascular age-related macular degeneration, intra-tympanic injection for sensorineural hearing loss, dermis injection for correction of wrinkles, creases and folds, signs of facial fat loss, volume loss, shallow to deep contour deficiencies, correction of depressed cutaneous scars, perioral rhytids, lip augmentation, facial lipoatrophy, stimulation of natural collagen production.

Crosslinked hydrogel compositions in accordance with the present disclosure include lubricious compositions for medical applications, compositions for therapeutic agent release (e.g., by including one or more therapeutic agents in a matrix of the crosslinked hydrogel), and implants (which may be formed ex vivo or in vivo) (e.g., compositions for use as tissue markers, compositions that act as spacers to reduce side effects of off-target radiation therapy, cosmetic compositions, etc.).

1. A multi-arm polymer comprising a functional core region and three or more polymer arms extending from the functional core region, each polymer arm of the three or more polymer arms comprising a hydrophilic polymer segment, a reactive moiety and a functional moiety, wherein the reactive moiety and the functional moiety are positioned at an end of each polymer arm and are covalently attached to the hydrophilic polymer segment.

2. The multi-arm polymer of claim 1, wherein the functional core region is an iodinated core region and wherein the functional moiety is an iodinated moiety.

3. The multi-arm polymer of claim 2, wherein the iodinated core region comprises a residue of an iodinated polyhydroxy compound.

4. The multi-arm polymer of claim 2, wherein the reactive moiety is covalently linked to the hydrophilic polymer segment through a linkage that comprises an ester group.

5. The multi-arm polymer of claim 2, wherein the reactive moiety is covalently linked to the hydrophilic polymer segment through a residue of an iodinated compound.

6. The multi-arm polymer of claim 2, wherein the reactive moiety is covalently linked to the hydrophilic polymer segment through a residue of an iodinated cyclic anhydride.

7. The multi-arm polymer of claim 2, wherein the reactive moiety is covalently linked to the hydrophilic polymer segment through a residue of a cyclic anhydride and a residue of an iodinated hydroxy acid compound.

8. The multi-arm polymer of claim 2, wherein the reactive moiety is covalently linked to the hydrophilic polymer segment through a residue of an iodinated amino acid compound and a residue of a cyclic anhydride.

9. The multi-arm polymer of claim 1, wherein the reactive moiety is covalently linked to the hydrophilic polymer segment through an amino-alcohol residue.

10. The multi-arm polymer of claim 1, wherein the reactive moiety comprises an electrophilic group.

11. The multi-arm polymer of claim 10, wherein the electrophilic group is a cyclic imide group.

12. The multi-arm polymer of claim 1, wherein the hydrophilic segment is selected from poly(alkylene oxide) segments, polysaccharide segments, polyoxazoline segments, polydioxanone segments, polypeptide segments, and polyvinyl alcohol segments.

13. A system comprising (a) a multi-arm polymer comprising a functional core region and three or more polymer arms extending from the functional core region, each polymer arm of the three or more polymer arms comprising a hydrophilic polymer segment, a reactive moiety and a functional moiety, wherein the reactive moiety and the functional moiety are positioned at an end of each polymer arm and are covalently attached to the hydrophilic polymer segment, and (b) a multifunctional compound comprising a plurality of reactive groups that are reactive with reactive moieties of the multi-arm polymer.

14. The system of claim 13, wherein the reactive moieties comprise electrophilic groups and the reactive groups are nucleophilic groups.

15. The system of claim 14, wherein the electrophilic groups are cyclic imide groups, and the nucleophilic groups are amino groups.

16. A method of treatment comprising administering to a subject a mixture that comprises (a) a multi-arm polymer comprising a functional core region and three or more polymer arms extending from the functional core region, each polymer arm of the three or more polymer arms comprising a hydrophilic polymer segment, a reactive moiety and a functional moiety, wherein the reactive moiety and the functional moiety are positioned at an end of each polymer arm and are covalently attached to the hydrophilic polymer segment, and (b) a multifunctional compound comprising a plurality of reactive groups that are reactive with reactive moieties of the multi-arm polymer, under conditions such that the multi-arm polymer and the multifunctional compound crosslink after administration to form a hydrogel.

17. The method of claim 16, wherein the method comprises administering to the subject a first fluid composition that comprises the multi-arm polymer and a second fluid composition that comprises the multifunctional compound.

18. The method of claim 16, wherein the method comprises administering to the subject a first fluid composition that comprises the multi-arm polymer and the multifunctional compound and a second fluid composition that comprises an accelerant that accelerates formation of covalent crosslinks between the multi-arm polymer and the multifunctional compound.

19. The method of claim 18, wherein the first fluid composition and the second fluid composition are delivered using a double barrel syringe.

20. A crosslinked hydrogel composition comprising a crosslinked reaction product of (a) the multi-arm polymer of claim 1 and (b) a multifunctional compound comprising a plurality of reactive groups that are reactive with reactive moieties of the multi-arm polymer, the reactive groups forming covalent bonds with the reactive moieties.
